(19) United States
(12) Patent Application Publication
(10) Pub. No.: US 2024/0392359 A1
(43) Pub. Date: Nov. 28, 2024

(54) DETECTION OF DONOR DNA IN TRANSPLANT RECIPIENT BIO-SAMPLE

(71) Applicant: ChromaCode, Inc., Carlsbad, CA (US)

(72) Inventors: Jeff Gole, Encinitas, CA (US); Lucien Jacky, Orange, CA (US); Gladys Ornelas, El Monte, CA (US); Chris MacDonald, San Diego, CA (US); Jerrod Schwartz, Carlsbad, CA (US); Andrew Richards, San Diego, CA (US)

(21) Appl. No.: 18/661,539

(22) Filed: May 10, 2024

Related U.S. Application Data
(60) Provisional application No. 63/465,824, filed on May 11, 2023.

(51) Int. Cl. C12Q 1/6851; C12Q 1/6858

(52) U.S. Cl. CPC C12Q 1/6851; C12Q 1/6858; C12Q 2600/156

(57) ABSTRACT

Described herein are methods, systems, compositions, and macromolecule complexes, for detecting, analyzing, evaluating, screening for, prognosing, diagnosing, and/or monitoring, of donor DNA in a transplant recipient for possible transplant rejection in the transplant recipient.

### CROSS REFERENCE TO RELATED APPLICATIONS

This application claims the benefit under 35 U.S.C. § 119(e) of U.S. Provisional Application No. 63/465,824, filed May 11, 2023, the content of which is incorporated by reference in its entirety into the present disclosure.

### TECHNICAL FIELD

Methods, systems, compositions, and macromolecule complexes, for detecting, analyzing, evaluating, screening for, prognosing, diagnosing, and/or monitoring, of donor DNA in a transplant recipient for possible transplant rejection in the transplant recipient.

### BACKGROUND

Methods, systems, compositions, and macromolecule complexes, for detecting, analyzing, evaluating, screening for, prognosing, diagnosing, and/or monitoring, donor DNA in a transplant recipient are important for transplant rejection. Because these methods, systems, compositions, and macromolecule complexes are not optimal, there is a need in the field for improved methods, systems, and compositions.

### SUMMARY

Described herein are novel methods, systems, compositions, and macromolecule complexes, for detecting, analyzing, evaluating, screening for, prognosing, diagnosing, and/or monitoring, donor DNA in a transplant recipient for possible transplant rejection.

In certain embodiments, the method for monitoring, detecting and/or diagnosing a transplant rejection in a patient described herein comprise the steps of: (a) isolating a cell-free DNA (cfDNA) from a plasma sample from the patient; (b) enriching a group of target regions in the cfDNA; (c) detecting a sequence encoding the SNP using a master mix; (d) determining a relative copy number for each target region; (e) identifying donor informative SNPs based on the relative copy number of each target relative to all other targets; (e) analyzing the donor informative SNP counts across all reactions to determine the total level of donor-derived cfDNA present; and (h) determining a rejection probability based on thresholds set based on background recipient-derived cfDNA.

In certain embodiments, the group of target regions in the cfDNA comprises from about 24 to about 384 target regions. In certain embodiments, the group of target regions in the cfDNA comprises from about 24 to 100 target regions. In certain embodiments, the group of target regions in the cfDNA comprises from about 24 to about 96 target regions. In certain embodiments, the group of target regions in the cfDNA comprises from about 24 to about 72 target regions. In certain embodiments, the group of target regions in the cfDNA comprises from about 24 to about 48 target regions. In certain embodiments, each target region comprise a single nucleotide polymorphism (SNP).

In certain embodiments, the group is predetermined for the patient without an a priori knowledge of a genotype of the patient. In certain embodiments, the group is predetermined for the patient without an a priori knowledge of a genotype of a transplant donor.

In certain embodiments, the plasma sample is isolated from a whole blood sample from the patient. In certain embodiments, the amplification bias is minimized. In certain embodiments, all target regions amplify with similar efficiencies.

In certain embodiments, the enriching step (b) comprise a ligation step. In certain embodiments, the enriching step (b) comprise a hybridization capture step. In certain embodiments, the enriching step (b) comprise a PCR amplification step. In certain embodiments, the enriching step (b) comprise a molecular inversion probes step. In certain embodiments, the enriching step (b) comprise a selective circularization step. In certain embodiments, the enriching step (b) is optionally split into sub-reactions. In certain embodiments, this optional step improves uniformity of the method.

In certain embodiments, the detecting sequence encoding the SNP in step (c) uses a master mix. In certain embodiments, the master mix is designed to detect N_{SNP}, wherein N_{SNP} is the number of sequences encoding a unique SNP. In certain embodiments, N_{SNP} is equal to or greater than the number of color channels on the detection instrument. In certain embodiments, a plurality of master mixes are processed simultaneously in a plurality of reactions and each master mix measures a plurality of different SNP. In certain embodiments, a plurality of master mixes are processed simultaneously. In certain embodiments, from about 2 to about 48 master mixes are processed simultaneously in a plurality of reactions. In certain embodiments, from about 2 to about 24 master mixes are processed simultaneously. In certain embodiments, from about 2 to about 12 master mixes are processed simultaneously. In certain embodiments, from about 4 to about 12 master mixes are processed simultaneously. In certain embodiments, from about 6 to about 12 master mixes are processed simultaneously. In certain embodiments, 8 master mixes are processed simultaneously.

In certain embodiments, the plurality of master mixes are processed simultaneously in a plurality of reactions. In certain embodiments, the plurality of master mixes are processed simultaneously in from about 4 to about 96 reactions. In certain embodiments, the plurality of master mixes are processed simultaneously in from about 4 to about 48 reactions. In certain embodiments, the plurality of master mixes are processed simultaneously in from about 4 to about 24 reactions. In certain embodiments, the plurality of master mixes are processed simultaneously in from about 4 to about 16 reactions. In certain embodiments, the plurality of master mixes are processed simultaneously in from about 4 to about 8 reactions. In certain embodiments, the plurality of master mixes are processed simultaneously in 8 reactions.

In certain embodiments, the sequence is amplified, preferably by a PCR, more preferably by a digital PCR, more preferably by a multiplex digital PCR.

In certain embodiments, prior to the sequences being amplified, the method further comprises a cfDNA quality control step. In certain embodiments, the quality control step comprises quantification and sizing of the cfDNA.

In certain embodiments, each target region comprise a single nucleotide polymorphism (SNP). In certain embodiments, the target regions comprise at least one informative targets. In certain embodiments, each informative target comprise an informative SNP. In certain embodiments, an informative SNP is present in the transplant donor and absent in the transplant patient. In certain embodiments, the group of target regions comprise at least two informative targets. In certain embodiments, the group of target regions comprise at least four informative targets. In certain embodiments, the group of target regions comprise at least six informative targets. In certain embodiments, the group of target regions comprise at least eight informative targets. In certain embodiments, the group of target regions comprise at least ten informative targets. In certain embodiments, the group of target regions comprise from about two to about 384 informative targets. In certain embodiments, the group of target regions comprise from about four to about 384 informative targets. In certain embodiments, the group of target regions comprise from about six to about 384 informative targets. In certain embodiments, the group of target regions comprise from about eight to about 384 informative targets. In certain embodiments, the group of target regions comprise from about ten to about 384 informative targets. In certain embodiments, the number of informative targets is achieved in an assay comprising at most 24 target regions. In certain embodiments, the number of informative targets is achieved in an assay comprising at most 48 target regions. In certain embodiments, the number of informative targets is achieved in an assay comprising at most 72 target regions. In certain embodiments, the number of informative targets is achieved in an assay comprising at most 96 target regions.

In certain embodiments, the SNP has a minor allele frequency in the human population of from about 0.1 to 0.39, from about 0.11 to 0.39, from about 0.12 to 0.39, from about 0.13 to 0.39, from about 0.14 to 0.39, from about 0.15 to 0.39, from about 0.16 to 0.39, from about 0.17 to 0.39, from about 0.18 to 0.39, from about 0.19 to 0.39, from about 0.2 to 0.39, from about 0.21 to 0.39, from about 0.22 to 0.39, from about 0.23 to 0.39, from about 0.24 to 0.39, from about 0.25 to 0.39, from about 0.26 to 0.39, from about 0.27 to 0.39, from about 0.28 to 0.39, from about 0.29 to 0.39, from about 0.3 to 0.39, from about 0.31 to 0.39, from about 0.32 to 0.39, from about 0.33 to 0.39, from about 0.34 to 0.39, from about 0.35 to 0.39, from about 0.36 to 0.39, from 0.37 to 0.39, and/or from 0.38 to 0.39.

In certain embodiments, the SNP has a minor allele frequency in the human population of from about 0.1 to 0.39, from about 0.1 to 0.38, from about 0.1 to 0.37, from about 0.1 to about 0.36, from about 0.1 to about 0.35, from about 0.1 to about 0.34, from about 0.1 to about 0.33, from about 0.1 to about 0.32, from about 0.1 to about 0.31, from about 0.1 to about 0.30, from about 0.1 to about 0.29, from about 0.1 to about 0.28, from about 0.1 to about 0.27, from about 0.1 to about 0.26, from about 0.1 to about 0.25, from about 0.1 to about 0.24, from about 0.1 to about 0.23, from about 0.1 to about 0.22, from about 0.1 to about 0.21, from about 0.1 to about 0.20, from about 0.1 to about 0.19, from about 0.1 to about 0.18, from about 0.1 to about 0.17, from about 0.1 to about 0.16, from about 0.1 to about 0.15, from about 0.1 to about 0.14, from about 0.1 to about 0.13, from about 0.1 to about 0.12, and/or from about 0.1 to about 0.11.

In certain embodiments, the SNP has a minor allele frequency in the human population of from about 0.1 to 0.39, from about 0.11 to 0.38, from about 0.12 to 0.37, from about 0.13 to about 0.36, from about 0.14 to about 0.35, from about 0.15 to about 0.34, from about 0.16 to about 0.33, from about 0.17 to about 0.32, from about 0.18 to about 0.31, from about 0.19 to about 0.30, from about 0.2 to about 0.29, from about 0.21 to about 0.28, from about 0.22 to about 0.27, from about 0.23 to about 0.26, and/or from about 0.24 to about 0.25.

In certain embodiments, the SNP has a minor allele frequency in the human population of about 0.3, about 0.29, about 0.293, or about 0.2929.

In certain embodiments, the SNP has a minor allele frequency in the human population of about 1−\sqrt{0.5}.

In certain embodiments, the SNP value is selected from a publication from a regional or a national institution or organization. In certain embodiments, the SNP value is selected from a publication from the National Center for Biotechnology Information, National Human Genome Research Institute, 1000 Genome Project, The Genome Aggregation Database (gnomAD), HapMap project, SNPedia, UK Biobank, and/or the likes.

In certain embodiments, the organ and/or tissue transplanted is a whole or a part of a kidney, liver, heart, lung, pancreas, intestine, stomach, cornea, heart valve, skin, bone marrow, bone, muscles, blood vessels, nerves, connective tissues, tendons, and/or combinations thereof. In certain embodiments, the organ and/or tissue transplanted is a whole or a part of a kidney, liver, heart, lung, pancreas, intestine, and/or combinations thereof. In certain embodiments, the organ and/or tissue transplanted is a whole or a part of a kidney, liver, heart, and/or combinations thereof. In certain embodiments, the organ and/or tissue transplanted is a whole or a part of a kidney. In certain embodiments, the organ and/or tissue transplanted is a whole or a part of a liver. In certain embodiments, the organ and/or tissue transplanted is a whole or a part of a heart.

In certain embodiments, the transplant donor is not a merozygote twin of the patient. In certain embodiments, the transplant donor does not have cancer at the time of transplant. In certain embodiments, the transplant donor is not pregnant at the time of transplant. In certain embodiments, the transplant donor the transplant donor is a full biological sibling of the patient. In certain embodiments, the transplant donor the transplant donor is a biological parent or biological child of the patient. In certain embodiments, the transplant donor the transplant donor is a half biological sibling of the patient. In certain embodiments, the transplant donor the transplant donor is a biologically unrelated to the patient.

In certain embodiments, the method further comprising isolating buffy coat gDNA in parallel for host genotyping. In certain embodiments, the buffy coat gDNA sample is processed through the same set of multiplex reactions to determine the host genotype. In certain embodiments, donor informative SNPs are identified based on a buffy coat gDNA genotype profile. In certain embodiments, a buffy coat dPCR signal is used in a background subtraction step to reduce noise.

In certain embodiments, the composition is used for monitoring a transplant rejection in a patient. In certain embodiments, the composition comprise a plurality of primers. In certain embodiments, the primer encodes a sequence complementary a target region. In certain embodiments, the plurality of primers has a total binding capacity to a group of target regions. In certain embodiments, the group of target regions has a total of from about 24 to about 384 target regions. In certain embodiments, the group of target regions has a total of from about 24 to 100 target regions. In certain embodiments, the group of target regions has a total of from about 24 to about 96 target regions. In certain embodiments, the group of target regions has a total of from about 24 to about 72 target regions. In certain embodiments, the group of target regions has a total of from about 24 to about 48 target regions. In certain embodiments, each target region comprise a single nucleotide polymorphism (SNP).

In certain embodiments, each SNP has a minor allele frequency in the world human population of from about 0.1 to 0.39, from about 0.11 to 0.39, from about 0.12 to 0.39, from about 0.13 to 0.39, from about 0.14 to 0.39, from about 0.15 to 0.39, from about 0.16 to 0.39, from about 0.17 to 0.39, from about 0.18 to 0.39, from about 0.19 to 0.39, from about 0.2 to 0.39, from about 0.21 to 0.39, from about 0.22 to 0.39, from about 0.23 to 0.39, from about 0.24 to 0.39, from about 0.25 to 0.39, from about 0.26 to 0.39, from about 0.27 to 0.39, from about 0.28 to 0.39, from about 0.29 to 0.39, from about 0.3 to 0.39, from about 0.31 to 0.39, from about 0.32 to 0.39, from about 0.33 to 0.39, from about 0.34 to 0.39, from about 0.35 to 0.39, from about 0.36 to 0.39, from 0.37 to 0.39, and/or from 0.38 to 0.39.

In certain embodiments, each SNP has a minor allele frequency in the world human population of from about 0.1 to 0.39, from about 0.1 to 0.38, from about 0.1 to 0.37, from about 0.1 to about 0.36, from about 0.1 to about 0.35, from about 0.1 to about 0.34, from about 0.1 to about 0.33, from about 0.1 to about 0.32, from about 0.1 to about 0.31, from about 0.1 to about 0.30, from about 0.1 to about 0.29, from about 0.1 to about 0.28, from about 0.1 to about 0.27, from about 0.1 to about 0.26, from about 0.1 to about 0.25, from about 0.1 to about 0.24, from about 0.1 to about 0.23, from about 0.1 to about 0.22, from about 0.1 to about 0.21, from about 0.1 to about 0.20, from about 0.1 to about 0.19, from about 0.1 to about 0.18, from about 0.1 to about 0.17, from about 0.1 to about 0.16, from about 0.1 to about 0.15, from about 0.1 to about 0.14, from about 0.1 to about 0.13, from about 0.1 to about 0.12, and/or from about 0.1 to about 0.11.

In certain embodiments, each SNP has a minor allele frequency in the world human population of from about 0.1 to 0.39, from about 0.11 to 0.38, from about 0.12 to 0.37, from about 0.13 to about 0.36, from about 0.14 to about 0.35, from about 0.15 to about 0.34, from about 0.16 to about 0.33, from about 0.17 to about 0.32, from about 0.18 to about 0.31, from about 0.19 to about 0.30, from about 0.2 to about 0.29, from about 0.21 to about 0.28, from about 0.22 to about 0.27, from about 0.23 to about 0.26, and/or from about 0.24 to about 0.25.

In certain embodiments, each SNP has a minor allele frequency in the world human population of about 0.3, about 0.29, about 0.293, or about 0.2929.

In certain embodiments, each SNP has a minor allele frequency in the world human population of about 1−\sqrt{0.5}.

In certain embodiments, the group of target regions comprise at least one informative targets. In certain embodiments, each informative target comprise an informative SNP. In certain embodiments, an informative SNP is present in the transplant donor and absent in the transplant patient. In certain embodiments, the group of target regions comprise at least two informative targets. In certain embodiments, the group of target regions comprise at least four informative targets. In certain embodiments, the group of target regions comprise at least six informative targets. In certain embodiments, the group of target regions comprise at least eight informative targets. In certain embodiments, the group of target regions comprise at least ten informative targets. In certain embodiments, the group of target regions comprise from about two to about 384 informative targets. In certain embodiments, the group of target regions comprise from about four to about 384 informative targets. In certain embodiments, the group of target regions comprise from about six to about 384 informative targets. In certain embodiments, the group of target regions comprise from about eight to about 384 informative targets. In certain embodiments, the group of target regions comprise from about ten to about 384 informative targets. In certain embodiments, the number of informative targets is achieved in an assay comprising at most 24 target regions. In certain embodiments, the number of informative targets is achieved in an assay comprising at most 48 target regions. In certain embodiments, the number of informative targets is achieved in an assay comprising at most 72 target regions. In certain embodiments, the number of informative targets is achieved in an assay comprising at most 96 target regions.

In certain embodiments, the informative SNP is bi-allelic. In certain embodiments, the sum of the two predominant allele frequency of the informative SNP is about 99% or greater in a population.

In certain embodiments, the plurality of primers is predetermined for the patient without an a priori knowledge of a genotype of the patient. In certain embodiments, the plurality of primers is predetermined for the patient without an a priori knowledge of a genotype of the transplant donor. In certain embodiments, the organ and/or tissue transplanted is a whole or a part of a kidney, liver, heart, lung, pancreas, intestine, stomach, cornea, heart valve, skin, bone marrow, bone, muscles, blood vessels, nerves, connective tissues, tendons, and/or combinations thereof. In certain embodiments, the organ and/or tissue transplanted is a whole or a part of a kidney, liver, heart, lung, pancreas, intestine, and/or combinations thereof. In certain embodiments, the organ and/or tissue transplanted is a whole or a part of a kidney, liver, heart, and/or combinations thereof. In certain embodiments, the organ and/or tissue transplanted is a whole or a part of a kidney. In certain embodiments, the organ and/or tissue transplanted is a whole or a part of a liver. In certain embodiments, the organ and/or tissue transplanted is a whole or a part of a heart.

In certain embodiments, the transplant donor is not a merozygote twin of the patient. In certain embodiments, the transplant donor does not have cancer at the time of transplant. In certain embodiments, the transplant donor is not pregnant at the time of transplant. In certain embodiments, the transplant donor is a full biological sibling of the patient. In certain embodiments, the transplant donor is a biological parent or biological child of the patient. In certain embodiments, the transplant donor is a half biological sibling of the patient. In certain embodiments, the transplant donor is biologically unrelated to the patient. In certain embodiments, the transplant organ or tissue is from a human.

In certain embodiments, the composition further comprises one or more of:
(a) a DNA polymerase; and/or
(b) a RNase H; and/or
(c) a dNTP mixture; and/or
(d) a buffer; and/or
(e) a detection probe
(f) a magnesium compound.

In certain embodiments, (a)-(f) of the composition are stored in separate compartments prior to use.

In certain embodiments, each detection probe comprises a fluorophore and optionally a quencher. In certain embodiments, the fluorophore and optionally the quencher is conjugated to the primer. In certain embodiments, each detection probe encodes a sequence complementary to the primer. In certain embodiments, the number of unique detection probes specific for each target region is between 1 and np, wherein np is selected from the list consisting of 1, 2, 3, 4, 5, 6, 7, 8, 9, 10, 11, and 12. In certain embodiments, the number of unique detection probes specific for the group of target regions in a sample volume is between 1 and Np, wherein Np is selected from the list of 1, 4, 8, 12, 16, 20, 24, 28, 32, 36, 40, 44, 48, 52, 56, 60, 64, 68, 72, 76, 80, 84, 88, 92 and 96 or 2, 3, 4, 5, 6, 7, 8, 9, 10, 11, 12, 13, 14, 15, 16, 17, 18, 19, and 20. In certain embodiments, a detection probe is conjugated to a first fluorophore and optionally conjugated to a first quencher. In certain embodiments, the combination of the emission color and emission intensity for each detection probe is unique.

In certain embodiments, the fluorophore is ABY, Alexa Fluor 350, Alexa Fluor 405, Alexa Fluor 488, Alexa Fluor 532, Alexa Fluor 546, Alexa Fluor 555, Alexa Fluor 568, Alexa Fluor 594, Alexa Fluor 647, AlexaFluor 680, Alexa Fluor 750, ATTO 425, ATTO 550, ATTO 590, Cyan500, Cy3, Cy5, Cy5.5, Texas Red, Fluorescein (FITC), 6-FAM, 5-FAM, HEX, JOE, TAMRA, ROX, BODIPY FL, Pacific Blue, Pacific Green, Coumarin, Oregon Green, Pacific Orange, VIC, LC610, CFR610, JA270, LC640, JUN, Trimethylrhodamine (TRITC), Cal Fluor dyes, Quasar dyes, DAPI, APC, Cyan Fluorescent Protein (CFP), Green Fluorescent Protein (GFP), Red Fluorescent Protein (RFP), Phycoerythin (PE), quantum dots (for example, Qdot 525, Qdot 565, Qdot 605, Qdot 705, Qdot 800), Cal Fluor Orange 560, Cal Fluor Red 590, Cal Fluor Red 610, Cal Fluor Red 635, Quasar 570, Quasar 670, Quasar 705, derivatives thereof and combinations thereof.

In certain embodiments, the fluorophore is selected from a group consisting of ATTO 425, FAM, HEX, TAMRA, Texas Red, Cy5, ATTO 590, ROX, or Cy5.5, derivatives thereof, and combinations thereof. In certain embodiments, the fluorophore is selected from a group of ATTO 425, FAM, HEX, Texas Red, Cy5, Cy5.5, derivatives thereof, and combinations thereof.

In certain embodiments, the quencher is a suitable quencher to the fluorophore. In certain embodiments, the quencher is TAMRA, BHQ-1, BHQ-2, BHQ-3, IowaBlack FQ, ZEN, or Dabcy, derivatives thereof, and combinations thereof.

In certain embodiments, the composition, once combined with a cell free DNA (cfDNA) from a plasma sample from the patient, can monitoring, detecting and/or diagnosing a transplant rejection in a patient.

In certain embodiments, the composition can amplify target regions, preferably by a PCR, more preferably by a digital PCR, more preferably by a multiplex digital PCR.

In certain embodiments, a kit is used for detecting, analyzing, evaluating, screening for, prognosing, diagnosing, and/or monitoring, a condition in the patient. In certain embodiments, the kit comprise the composition described herein. In certain embodiments, the kit comprise primers and one or more of (a)-(f) are stored separately in a container. In certain embodiments, the primers and two or more of (a)-(f) are pre-mixed and stored together in a container. In certain embodiments, the kit further comprise an instruction for the method described herein.

### BRIEF DESCRIPTION OF THE DRAWINGS

FIG. 1 shows the pre-amplification bias in a single allele approach with 3000 copy input.

FIG. 2 shows the pre-amplification bias in dual allele approach with 3000 copy input.

FIG. 3 shows the percent coefficient of variation (% CV) adjusted loci ratio for sibling and unrelated individuals as a function of donor fraction.

FIG. 4 shows an example of simulated positive predictive value (PPV) and negative predictive value (NPV) for 3000 copies of input DNA.

FIG. 5 shows the expected versus the observed donor fraction for a single allele.

FIG. 6 shows the in silico projection of the likelihood that a 24 target assay, 48 target assay, 72 target assay, and 96 target assay would result in a minimum of 2, 4, 6, 8, and 10 informative targets, when the donor and recipient of the transplant are full siblings. The table also shows the mean and media number of informative targets in a 24 target assay, 48 target assay, 72 target assay, and 96 target assay. Each projection was generated from 10,000 simulated donor-recipient pairs for 66 populations with genotypic frequencies sampled from a common set of 92 loci from the ALFRED database. Loci where the recipient was homozygous for the major allele and the donor had a different genotype were called informative, simulating an assay that detects the minor allele.

FIG. 7 shows a bar chart of the number of informative markers for each relationship type when simulating all possible permutations of Donor-Recipient crosses of the individuals in the 1000 Genomes database. Using a theoretical assay that detects minor alleles for a set of 91 markers (the minor allele for each locus has a population mean allele frequency of ˜0.40).

FIG. 8 shows the signal generated by an assay to detect the T allele of rs987640 tested against cell-line samples at approximately 5000 copies per reaction. A cell line sample representing a recipient with an A/A genotype is not detected by the assay. The cell line representing a donor with a T/T genotype is detected at approximately 5000 copies. The mixed sample of 99% recipient and 1% donor cell lines detects the T allele at approximately 50 copies in a 4950 copy background. FIG. 9 shows the amount of cfDNA following a ligation-based pre-amplification reaction using 5 ng of cfDNA from 6 different donors in a 3 color 2 intensity level 6-plex dPCR for a reference allele and an alternative allele. Each reaction is performed in duplicates and the amount of cfDNA output is averaged. 6 SNPs are resolved and whether the allele was homozygous or heterozygous was determined.

FIG. 10 shows the amount of cfDNA following a ligation-based pre-amplification reaction using 17 ng of cfDNA from 2 different donors or 17 ng of a mixture of cfDNA from two different donors. A 3 color 2 intensity level 6-plex dPCR was performed, in duplicates, and the average amount of amplified cfDNA is recorded for each reference allele and the alternative allele. 6 SNPs are resolved and whether the allele was homozygous or heterozygous was determined.

FIG. 11 shows the amount of cfDNA following a targeted PCR pre-amplification reaction using 5 ng of cfDNA from 5 different donors in a 3 color 2 intensity level 6-plex dPCR for a reference allele and an alternative allele. Each reaction is performed in duplicates and the amount of cfDNA output is averaged. 6 SNPs are resolved and whether the allele was homozygous or heterozygous was determined.

FIG. 12 shows the amount of cfDNA following a targeted PCR pre-amplification reaction using 5 ng of cfDNA from 2 different donors or 5 ng of a mixture of cfDNA from two different donors. A 3 color 2 intensity level 6-plex dPCR was performed, in duplicates, and the average amount of amplified cfDNA is recorded for each reference allele and the alternative allele. 6 SNPs are resolved and whether the allele was homozygous or heterozygous was determined.

FIG. 13 shows the amount of cfDNA following a targeted PCR pre-amplification reaction using 17 ng of cfDNA from 2 different donors or 5 ng of a mixture of cfDNA from two different donors. A 3 color 2 intensity level 6-plex dPCR was performed, in duplicates, and the average amount of amplified cfDNA is recorded for each reference allele and the alternative allele. 6 SNPs are resolved and whether the allele was homozygous or heterozygous was determined

FIG. 14 shows results of a multiplex assay that measures both reference and alternative alleles at 40 unique genomic loci. The results are summarized as pooled median donor fraction of all markers called informative by the algorithm (calculated donor fraction between 0.25% and 10%, with more than 3 informative markers are required for a donor fraction call). Samples of unrelated blood donors samples mixed at 0%, 1%, and 3% “donor” to “recipient” sample. Two replicates of Subject Pair 1 and Subject Pair 2 are shown.

FIG. 15 shows the results of a multiplex assay that detects both reference and alternative alleles for 40 unique loci. The red dot is the pooled median donor fraction of all informative markers, and the grey dot is the donor fraction of the individual marker. The numbers above the points are the number of markers called informative for that sample by the algorithm (calculated donor fraction between 0.25% and 10%, more than 3 informative markers are required for a donor fraction call). Samples of unrelated blood donors samples were mixed at 0%, 1%, and 3% “donor” to “recipient”. Two replicates of Mix 1 and Mix 2 are shown.

FIG. 16 shows the Limit of Blank experiment using a 40 loci multiplex assay. Seventeen samples were tested. The points are the fraction of each individual marker for a sample. The numbers above the points are the total number of markers called informative for that sample by the algorithm (calculated donor fraction between 0.25 and 10%).

FIG. 17 shows a flow chart of the workflow from blood draw to analysis.

### DETAILED DESCRIPTION

### Interpretations and Definitions

Unless otherwise indicated, this description employs conventional chemical, biochemical, biotechnology, clinical biotechnology, molecular biology, immunology, cancer biology, clinical medicine, and pharmacology methods and terms that have their ordinary meaning to persons of skill in this field (unless otherwise defined/described herein). All publications, references, patents and patent applications cited herein are hereby incorporated by reference in their entireties.

As used in this specification and the appended claims, the following general rules apply. Singular forms “a,” “an” and “the” include plural references unless the content clearly indicates otherwise.

As used herein, the following terms shall have the specified meaning. The term “about” takes on its plain and ordinary meaning of “approximately” as a person of skill in the art would understand, and unless specified otherwise, means plus or minus 10% of a value. The term “comprise,” “comprising,” “contain,” “containing,” “include,” “including,” “include but not limited to,” or “characterized by” is inclusive or open-ended and does not exclude additional, unrecited elements.

As used herein, the following terms shall have the specified meaning.

“Allele” means a variation of a gene at a specific genome location.

“Allele fraction” means the fraction, or frequency, of an allele at a specific locus in a population.

“Dilution noise” means the noise introduced by using dilution. For example, a template DNA can be diluted to a concentration of 1 molecule per partition or per PCR reaction. As another example, the concentration of a template DNA can also be diluted to a desired concentration of #copies/μL solution, such that when a volume of the solution is loaded into a well, the desired template DNA per well is achieved.

“dPCR” means digital polymerase chain reaction. dPCR is a technique capable of detecting single nucleotide variants, chromosomal translocations, allelic imbalance, and differences in gene expression.

“dPCR noise” means the noise introduced by an dPCR instrument during a dPCR process.

“Extraction noise” means the noise introduced when extracting DNA from a whole blood.

“Multiplex” means the process of targeting multiple alleles within a single PCR mixture through various primers and fluorescent probes.

“Poisson noise” means the Poisson distribution used to simulate the proportions of positive partitions containing the targets generated by a dPCR instrument.

“Pre-amplification bias” means the skewed, or unbalanced, amplification of target samples that may occur during pre-amplification. Pre-amplification prior to dPCR is often necessary when working with low concentrations.

“SNP” means single nucleotide polymorphisms. Single nucleotide polymorphism is a variation in a DNA sequence that occurs when a single nucleotide is altered in the genome sequence.

“Informative target” refers to a target SNP allele that is present in the donor genome, is detectable by the assay, and that target SNP allele is not present in the recipient's genome.

“Bi-allelic” means the two predominant alleles in a general population. Generally a population is bi-allelic if the combined frequency of the two predominant alleles is about 99% or more in the population.

### Transplant Rejection

Organ transplantation is a lifesaving procedure, particularly for patients with end-stage organ failure. However, transplant rejection continues to be a major challenge for transplant recipients. Two of the major contributors for transplant rejection include irreversible chronic rejection and immunosuppression complications, such as nephrotoxicity, cardiovascular disease, opportunistic infection, and malignancy. It is estimated that more than 50% of transplanted kidneys from deceased donors fail within 10 years.

Currently, monitoring for transplant rejection primarily involves the use of invasive biopsy procedures, which carry risks of serious complications (see, for example, Schwarz A et al. (2005), Limaye A R et al. (2012), and Eikmans M et al. (2019). Additionally, while noninvasive markers exist and sequencing based approaches are promising (see, for example, Beck et al., (2013), Beck et al., (2015), and Beck et al., (2018), Snyder (2011), Grskovic (2016), Huang (2019), Kim (2022), Marsh (2019), North (2020), and Sigdel (2019), problems exist. For example, Clausen F B et al., (2023)) when using the design and set up from Beck et al., (2013), Beck et al., (2015), and Beck et al., (2018), more than half (22 out of 18) assays were discarded because it did not meet one or more of the in silico verification and quality assurance selections, such as checking correct DNA sequences, potential overlap of primers and probes, melting temperatures, and potential secondary structure formation. As a result, Clausen F B et al., (2023) designed bi-allelic SNP targets with minor allele frequency of 0.4 to 0.5 (Rs numbers with MAF 0.4-0.5) and with no additional SNPs of >1% frequency present in the primer or probe sequence, and with a maximum amplicon length of 90 bp.

Additionally, existing methods, such as those described in Beck et al., (2018) is a two-step process. The first step is to identify the homozygous SNPs in the transplant recipient, which can involve as many as 38 assays. The second step is to use the set of homologous SNPs to identify assays showing an allele that is heterologous to the recipient. In these methods, all subsequent samples are tested only with the patient's personal informative assay set. Thus, these two-step processes uses patient-specific genetic information and relies on a step to predetermine the donor and patient genotypes for each assay. Since these assays, by design, requires informed patient samples and a design that is based on transplant-recipient genotypes, the assays can be costly, can require a specialized centralized facility to conduct these complex assays, and can be more prone to errors. Thus, the existing technologies may not always provide an accurate assessment of the graft status. There is a need for non-invasive and accurate methods to monitor transplant recipients for signs of organ rejection. Moreover, there is a critical need for a single-step, agnostic, easily adaptable, non-invasive assay to detect transplant rejection for any transplant patient.

In order to develop such an assay, three key parameters have to be considered: 1) clinical utility, 2) patient population and sample compatibility, and 3) simplicity of adoption. First, the assay should have meaningful impact on clinical decision making such that patient outcomes are improved. An assay that can be run locally, instead of in a central lab, could lead to potentially same-day results, which would avoid biopsy decision delays. Second, the assay should have utility for the majority of a population of interest and be amenable to require two or fewer tubes of blood. Human population genetics are such that 48 SNPs can resolve diverse populations with high accuracy and inclusivity. Coupled with Applicant's methods, these SNPs can be measured in a single instrument run, at low cost, on many commercial digital PCR instruments. Third, PCR instruments and assays are straightforward for laboratories to adopt and do not require complicated analysis pipelines.

Applicant has invented new methods, systems, compositions, and macromolecule complexes, for detecting, analyzing, evaluating, screening for, prognosing, diagnosing, and/or monitoring, donor DNA in a transplant recipient, using genetic characteristics of a population that the transplant recipient belongs to, and without determining the specific genetic sequences of the specific transplant patient or transplant donor. The innovations disclosed herein are important improvements on existing technologies because they do not require an priori knowledge of the genotype of the transplant recipient and/or donor. In certain embodiments, the inventions do not require donor genotypes to be identified. In certain embodiments, the inventions do not require recipient genotypes to be identified. In certain embodiments, the inventions do not require both donor and recipient genotypes to be identified. Unlike existing methods where patient and donor genotypes need to be predetermined, the embodiments of these single-step methods and systems, can be agnostic with respect to the recipient and donor genotypes throughout the entire detection, analysis, screening, prognosis, diagnosis, and/or monitor process. Thus, the superior methods, systems, compositions, and macromolecule complexes are simpler, more cost effective, and less prone to errors. Unlike traditional technology that require a centralized facility for processing the samples, the technology disclosed herein are easily adaptable, can be easily streamlined, and can be easily distributed and implemented in multiple labs.

### Cell-Free DNA

Cell-free DNA (cfDNA) are small fragments of DNA that are released into the bloodstream during cell death. Recent studies have shown that the level of donor-derived cfDNA (dd-cfDNA) in the recipient's blood can serve as a biomarker for transplant rejection. However the amount of dd-cfDNA in plasma can be extremely low, with some estimating it to be 10 genomic copies per mL of plasma (Beck et al., (2018). Thus, it has been a challenge to accurately measure and meaningful monitor transplant rejection using cfDNA, with applications typically limited to gender-mismatched donor-recipient rejections. HLA-mismatch techniques can improve accuracy of dd-cfDNA assays. However, HLA-mismatch techniques requires an a priori knowledge of the HLA genotypes and laboriously optimize each donor-recipient pair for the several hundred known HLA genotypes (see, e.g.: Beck et al., (2018)).

However, current methods for dd-cfDNA detection involve complex and expensive sequencing procedures. Given the need for requiring an a priori knowledge of donor and recipient genotypes, which may not always be available, there is a need for an improved method for dd-cfDNA detection that can overcome these limitations.

### SNP Mediated Monitoring of Transplant Rejection

Described herein are improved methods, systems, compositions, and macromolecule complexes, for detecting, analyzing, evaluating, screening for, prognosing, diagnosing, and/or monitoring, of donor DNA in a transplant recipient for possible transplant rejection. These improvements requires no a prior knowledge of the donor and recipient genotypes and does not require complex and expensive sequencing, which are typically associated with traditional technologies. In certain embodiments, highly multiplexed digital PCR are used. In certain embodiments, the methods, systems, compositions, and macromolecule complexes are mediated by single nucleotide polymorphisms (SNPs). In certain embodiments, highly multiplexed digital PCR simultaneously determine which SNPs are from the donor and which are from the recipient by looking at the quantity of each SNPs found, without an a priori knowledge of the donor and recipient genotypes. In certain embodiments, a model is used to mediate the determination, detecting, analyzing, evaluating, screening for, prognosing, diagnosing, and/or monitoring, of donor DNA in a transplant recipient for possible transplant rejection. In certain embodiments, the amount of donor DNA as measured by the pre-determined SNP are compared to the recipient DNA.

### SNP Allelic Frequencies

Traditional methods have determined that SNP should be preselected for having a minor allele frequency (MAF) of between 0.4 and 0.5 based on Hardy-Weinberg equilibrium calculation. That is, the minor allele would have a nearly equal distribution of both alleles in a given population (see, for example, Beck et al., (2018) and Clausen et al., (2023)). We have determined that the optimal allele frequency of the targeted allele in a bi-allelic SNP has a MAF of from about 0.1 to about 0.4. Allelic frequencies at this range can provide a higher proportion of informative loci per marker than detecting both alleles.

In certain embodiments the MAF has a range of from about 0.1 to 0.40, from about 0.1 to 0.39, from about 0.1 to 0.38, from about 0.1 to 0.37, from about 0.1 to about 0.36, from about 0.1 to about 0.35, from about 0.1 to about 0.34, from about 0.1 to about 0.33, from about 0.1 to about 0.32, from about 0.1 to about 0.31, from about 0.1 to about 0.30, from about 0.1 to about 0.29, from about 0.1 to about 0.28, from about 0.1 to about 0.27, from about 0.1 to about 0.26, from about 0.1 to about 0.25, from about 0.1 to about 0.24, from about 0.1 to about 0.23, from about 0.1 to about 0.22, from about 0.1 to about 0.21, from about 0.1 to about 0.20, from about 0.1 to about 0.19, from about 0.1 to about 0.18, from about 0.1 to about 0.17, from about 0.1 to about 0.16, from about 0.1 to about 0.15, from about 0.1 to about 0.14, from about 0.1 to about 0.13, from about 0.1 to about 0.12, and/or from about 0.1 to about 0.11.

In certain embodiments, the MAF has a range of from about 0.1 to 0.39, from about 0.11 to 0.39, from about 0.12 to 0.39, from about 0.13 to 0.39, from about 0.14 to 0.39, from about 0.15 to 0.39, from about 0.16 to 0.39, from about 0.17 to 0.39, from about 0.18 to 0.39, from about 0.19 to 0.39, from about 0.2 to 0.39, from about 0.21 to 0.39, from about 0.22 to 0.39, from about 0.23 to 0.39, from about 0.24 to 0.39, from about 0.25 to 0.39, from about 0.26 to 0.39, from about 0.27 to 0.39, from about 0.28 to 0.39, from about 0.29 to 0.39, from about 0.3 to 0.39, from about 0.31 to 0.39, from about 0.32 to 0.39, from about 0.33 to 0.39, from about 0.34 to 0.39, from about 0.35 to 0.39, from about 0.36 to 0.39, from 0.37 to 0.39, and/or from 0.38 to 0.39.

In certain embodiments, the MAF has a range of from about 0.1 to 0.39, from about 0.11 to 0.38, from about 0.12 to 0.37, from about 0.13 to about 0.36, from about 0.14 to about 0.35, from about 0.15 to about 0.34, from about 0.16 to about 0.33, from about 0.17 to about 0.32, from about 0.18 to about 0.31, from about 0.19 to about 0.30, from about 0.2 to about 0.29, from about 0.21 to about 0.28, from about 0.22 to about 0.27, from about 0.23 to about 0.26, and/or from about 0.24 to about 0.25.

In certain embodiments, the optimum allele frequency of the detected (or targeted) allele is 0.3, 0.29, 0.293, and/or 0.2929. In certain embodiments the optimum allele frequency of the detected (or targeted) allele is 1−\sqrt{0.5}.

For a marker to be informative in detecting donor derived cfDNA, the marker must be present in the donor and absent in the recipient. In unrelated donor recipient, when detecting a single minor allele, the range of expected ratio of informative markers per loci interrogated is from 0.154 to 0.23, for MAF ranges from 0.1 to 0.4. When detecting both the major and minor allele the range of expected ratio of informative markers per loci interrogated is from 0.164 to 0.365, for MAF ranges from 0.1 to 0.4. The expected ratio of informative markers to total markers decreases with degree of relatedness of the donor and recipient. Tables 1 and 2 below for more detailed estimates of the ratio of informative markers depending on donor-recipient relationships.

TABLE 1
The expected ratio of informative markers per loci
interrogated for single Minor allele detection method.

| MAF | unrelated | Full sibling | Half sibling | Parent-child |
|---|---|---|---|---|
| 0.1 | 0.154 | 0.079 | 0.117 | 0.081 |
| 0.11 | 0.165 | 0.0847 | 0.126 | 0.0871 |
| 0.12 | 0.175 | 0.0901 | 0.134 | 0.0929 |
| 0.13 | 0.184 | 0.0952 | 0.141 | 0.0984 |
| 0.14 | 0.193 | 0.0999 | 0.148 | 0.104 |
| 0.15 | 0.2 | 0.104 | 0.154 | 0.108 |
| 0.16 | 0.208 | 0.108 | 0.16 | 0.113 |
| 0.17 | 0.214 | 0.112 | 0.166 | 0.117 |
| 0.18 | 0.22 | 0.116 | 0.171 | 0.121 |
| 0.19 | 0.226 | 0.119 | 0.175 | 0.125 |
| 0.2 | 0.23 | 0.122 | 0.179 | 0.128 |
| 0.21 | 0.235 | 0.124 | 0.183 | 0.131 |
| 0.22 | 0.238 | 0.126 | 0.186 | 0.134 |
| 0.23 | 0.241 | 0.129 | 0.189 | 0.136 |
| 0.24 | 0.244 | 0.13 | 0.191 | 0.139 |
| 0.25 | 0.246 | 0.132 | 0.193 | 0.141 |
| 0.26 | 0.248 | 0.133 | 0.195 | 0.142 |
| 0.27 | 0.249 | 0.134 | 0.196 | 0.144 |
| 0.28 | 0.25 | 0.135 | 0.197 | 0.145 |
| 0.29 | 0.25 | 0.136 | 0.198 | 0.146 |
| 0.3 | 0.25 | 0.136 | 0.198 | 0.147 |
| 0.31 | 0.249 | 0.136 | 0.199 | 0.148 |
| 0.32 | 0.249 | 0.136 | 0.198 | 0.148 |
| 0.33 | 0.247 | 0.136 | 0.198 | 0.148 |
| 0.34 | 0.246 | 0.136 | 0.197 | 0.148 |
| 0.35 | 0.244 | 0.135 | 0.196 | 0.148 |
| 0.36 | 0.242 | 0.134 | 0.195 | 0.147 |
| 0.37 | 0.239 | 0.133 | 0.193 | 0.147 |
| 0.38 | 0.237 | 0.132 | 0.191 | 0.146 |
| 0.39 | 0.234 | 0.131 | 0.189 | 0.145 |
| 0.4 | 0.23 | 0.13 | 0.187 | 0.144 |

TABLE 2
Expected ratio of informative markers per loci interrogated for
both Major and Minor allele detection method.

| MAF | unrelated | Full sibling | Half sibling | Parent-child |
|---|---|---|---|---|
| 0.1 | 0.164 | 0.0859 | 0.127 | 0.09 |
| 0.11 | 0.177 | 0.0931 | 0.137 | 0.0979 |
| 0.12 | 0.189 | 0.1 | 0.147 | 0.106 |
| 0.13 | 0.201 | 0.107 | 0.157 | 0.113 |
| 0.14 | 0.212 | 0.113 | 0.166 | 0.12 |
| 0.15 | 0.222 | 0.119 | 0.175 | 0.128 |
| 0.16 | 0.233 | 0.125 | 0.184 | 0.134 |
| 0.17 | 0.242 | 0.131 | 0.192 | 0.141 |
| 0.18 | 0.252 | 0.137 | 0.2 | 0.148 |
| 0.19 | 0.26 | 0.142 | 0.207 | 0.154 |
| 0.2 | 0.269 | 0.147 | 0.214 | 0.16 |
| 0.21 | 0.277 | 0.152 | 0.221 | 0.166 |
| 0.22 | 0.284 | 0.157 | 0.228 | 0.172 |
| 0.23 | 0.291 | 0.161 | 0.234 | 0.177 |
| 0.24 | 0.298 | 0.166 | 0.24 | 0.182 |
| 0.25 | 0.305 | 0.17 | 0.246 | 0.188 |
| 0.26 | 0.311 | 0.174 | 0.252 | 0.192 |
| 0.27 | 0.317 | 0.178 | 0.257 | 0.197 |
| 0.28 | 0.322 | 0.181 | 0.262 | 0.202 |
| 0.29 | 0.327 | 0.185 | 0.266 | 0.206 |
| 0.3 | 0.332 | 0.188 | 0.271 | 0.21 |
| 0.31 | 0.336 | 0.191 | 0.275 | 0.214 |
| 0.32 | 0.341 | 0.194 | 0.279 | 0.218 |
| 0.33 | 0.344 | 0.197 | 0.283 | 0.221 |
| 0.34 | 0.348 | 0.199 | 0.286 | 0.224 |
| 0.35 | 0.351 | 0.202 | 0.289 | 0.227 |
| 0.36 | 0.355 | 0.204 | 0.293 | 0.23 |
| 0.37 | 0.358 | 0.206 | 0.295 | 0.233 |
| 0.38 | 0.36 | 0.208 | 0.298 | 0.236 |
| 0.39 | 0.363 | 0.21 | 0.3 | 0.238 |
| 0.4 | 0.365 | 0.211 | 0.302 | 0.24 |

Relative dd-cfDNA Indicative of a Transplant Failure

In certain embodiments, the relative percent of dd-cfDNA to total cfDNA in the plasma of an organ or tissue transplant patient experiencing a rejection as measured using the method, systems, compositions, and macromolecule complexes disclosed herein is from about 80% to about 90%, from about 75% to about 90%, from about 70% to about 90%, from about 65% to about 90%, from about 60% to about 90%, from about 55% to about 90%, from about 50% to about 90%, from about 45% to about 90%, from about 40% to about 90%, from about 35% to about 90%, from about 30% to about 90%, from about 25% to about 90%, from about 20% to about 90%, 15% to about 90%, from about 14% to about 90%, from about 13% to about 90%, from about 12% to about 90%, from about 11% to about 90%, about 10% to 90%, from about 9% to about 90%, from about 8.5% to about 90%, from about 7% to about 90%, from about 6% to about 90%, from about 5% to about 90%, from about 4% to about 90%, from about 3% to about 90%, from about 2% to about 90%, from about 1% to about 90%, from about 0.75% to about 90%, from about 0.65% to about 90%, from about 0.55% to about 90%, from about 0.45% to about 90%, from about 0.35% to about 90%, from about 0.25% to about 90%, from about 0.15% to about 90%, from about 0.05% to about 90%, from about 0.04% to about 90%, from about 0.03% to about 90%, from about 0.02% to about 90%, from about 0.01% to about 90% when measured at a post-transplant time of from about 5 days to about 3650 days, from about 7 days to about 3650 days, from about 14 days to about 3650 days, from about 30 days to about 3650 days, from about 45 days to about 3650 days, from about 60 days to about 3650 days, from about 75 days to about 3650 days, from about 90 days to about 3650 days, from about 105 days to about 3650 days, from about 120 days to about 3650 days, from about 135 days to about 3650 days, from about 150 days to about 3650 days, from about 165 days to about 3650 days, from about 180 days to about 3650 days, from about 195 days to about 3650 days, from about 210 days to about 3650 days, from about 225 days to about 3650 days, from about 240 days to about 3650 days, from about 255 days to about 3650 days, from about 270 days to about 3650 days, from about 285 days to about 3650 days, from about 300 days to about 3650 days, from about 315 days to about 3650 days, from about 330 days to about 3650 days, from about 345 days to about 3650 days, from about 360 days to about 3650 days.

In certain embodiments, the relative percent of dd-cfDNA to total cfDNA in the plasma of a liver transplant patient experiencing an organ rejection as measured using the method, systems, compositions, and macromolecule complexes disclosed herein is from about 60% to about 90%, from about 55% to about 90%, from about 50% to about 90%, from about 45% to about 90%, from about 40% to about 90%, from about 35% to about 90%, from about 30% to about 90%, from about 25% to about 90%, from about 20% to about 90%, 15% to about 90%, from about 14% to about 90%, from about 13% to about 90%, from about 12% to about 90%, from about 11% to about 90%, about 10% to 90%, from about 9% to about 90%, from about 8.5% to about 90%, from about 7.5% to about 90%, from about 6.5% to about 90%, from about 5.5% to about 90%, from about 4.5% to about 90%, and/or from about 3.5% to about 90% when measured at a post-transplant time of from about 5 days to about 3650 days, from about 7 days to about 3650 days, from about 14 days to about 3650 days, from about 30 days to about 3650 days, from about 45 days to about 3650 days, from about 60 days to about 3650 days, from about 75 days to about 3650 days, from about 90 days to about 3650 days, from about 105 days to about 3650 days, from about 120 days to about 3650 days, from about 135 days to about 3650 days, from about 150 days to about 3650 days, from about 165 days to about 3650 days, from about 180 days to about 3650 days, from about 195 days to about 3650 days, from about 210 days to about 3650 days, from about 225 days to about 3650 days, from about 240 days to about 3650 days, from about 255 days to about 3650 days, from about 270 days to about 3650 days, from about 285 days to about 3650 days, from about 300 days to about 3650 days, from about 315 days to about 3650 days, from about 330 days to about 3650 days, from about 345 days to about 3650 days, from about 360 days to about 3650 days.

In certain embodiments, the relative percent of dd-cfDNA to total cfDNA in the plasma of a kidney transplant patient experiencing an organ rejection as measured using the method, systems, compositions, and macromolecule complexes disclosed herein is from about 10% to 90%, from about 9% to about 90%, from about 8% to about 90%, from about 7% to about 90%, from about 6% to about 90%, from about 5% to about 90%, from about 4% to about 90%, from about 3% to about 90%, from about 2% to about 90%, from about 1% to about 90%, from about 0.75% to about 90%, and/or from about 0.5% to about 90% when measured at a post-transplant time of from about 5 days to about 3650 days, from about 7 days to about 3650 days, from about 14 days to about 3650 days, from about 30 days to about 3650 days, from about 45 days to about 3650 days, from about 60 days to about 3650 days, from about 75 days to about 3650 days, from about 90 days to about 3650 days, from about 105 days to about 3650 days, from about 120 days to about 3650 days, from about 135 days to about 3650 days, from about 150 days to about 3650 days, from about 165 days to about 3650 days, from about 180 days to about 3650 days, from about 195 days to about 3650 days, from about 210 days to about 3650 days, from about 225 days to about 3650 days, from about 240 days to about 3650 days, from about 255 days to about 3650 days, from about 270 days to about 3650 days, from about 285 days to about 3650 days, from about 300 days to about 3650 days, from about 315 days to about 3650 days, from about 330 days to about 3650 days, from about 345 days to about 3650 days, from about 360 days to about 3650 days.

In certain embodiments, the relative percent of dd-cfDNA to total cfDNA in the plasma of a heart transplant patient experiencing an organ rejection as measured using the method, systems, compositions, and macromolecule complexes disclosed herein is from about 5% to about 90%, from about 4% to about 90%, from about 3% to about 90%, from about 2% to about 90%, from about 1% to about 90%, from about 0.75% to about 90%, from about 0.65% to about 90%, from about 0.55% to about 90%, from about 0.45% to about 90%, from about 0.35% to about 90%, from about 0.25% to about 90%, from about 0.17% to about 90%, and/or from about 0.15% to about 90% when measured at a post-transplant time of from about 5 days to about 3650 days, from about 7 days to about 3650 days, from about 14 days to about 3650 days, from about 30 days to about 3650 days, from about 45 days to about 3650 days, from about 60 days to about 3650 days, from about 75 days to about 3650 days, from about 90 days to about 3650 days, from about 105 days to about 3650 days, from about 120 days to about 3650 days, from about 135 days to about 3650 days, from about 150 days to about 3650 days, from about 165 days to about 3650 days, from about 180 days to about 3650 days, from about 195 days to about 3650 days, from about 210 days to about 3650 days, from about 225 days to about 3650 days, from about 240 days to about 3650 days, from about 255 days to about 3650 days, from about 270 days to about 3650 days, from about 285 days to about 3650 days, from about 300 days to about 3650 days, from about 315 days to about 3650 days, from about 330 days to about 3650 days, from about 345 days to about 3650 days, from about 360 days to about 3650 days.

### Biological Samples Containing Nucleic Acid Targets/Nucleic Acid Analytes

A nucleic acid target, also referred to as a nucleic acid analyte of the present disclosure may be derived from a sample (i.e. a biological sample). A biological sample for the method, systems, compositions, and macromolecule complexes disclosed herein may be a sample derived from a subject. In certain embodiments, a subject is a human subject.

A sample collected from the subject may comprise any number of macromolecules, for example, cellular macromolecules. A biological sample may contain nucleic acid analyte from the subject described herein, and a second nucleic acid analyte from a source different from the subject. In certain embodiments, the subject is a recipient of an tissue and/or organ transplant. In certain embodiments, the source is a donor organism, including a donor of tissue and/or organ transplant. In certain embodiments, the donor organism is a human.

A biological sample may be a fluid sample, including a blood sample, plasma sample, urine sample, or saliva sample. A biological sample may be, for example, blood, plasma, serum, urine, saliva, mucosal excretions, sputum, stool or tears. The sample may be a collection of samples. For example, a sample may be pooled with other sample and then subjected to methods described elsewhere herein.

A nucleic acid target may be derived from one or more cells. A nucleic acid target may comprise deoxyribonucleic acid (DNA). DNA may be any kind of DNA, including genomic DNA. A nucleic acid target may be viral DNA. A nucleic acid target may comprise ribonucleic acid (RNA). RNA may be any kind of RNA, including messenger RNA, transfer RNA, ribosomal RNA, and microRNA. RNA may be viral RNA. The nucleic acids may comprise a human genomic sequence. The nucleic acid may comprise a wild type sequence. The nucleic acid may comprise a variant sequence.

Nucleic acid targets may comprise one or more members. A member may be any region of a nucleic acid target. A member may be of any length. A member may be, for example, up to 1, 2, 3, 4, 5, 10, 20, 50, 100, 500, 1000, 5000, 10000, 50000, or 100000 nucleotides, or more. In some instances, a member may be a gene. A nucleic acid target may comprise a gene whose detection may be useful in diagnosing one or more transplant and/or tissue rejection in a subject. The gene may comprise one or more single nucleotide polymorphisms (SNPs). The SNP may have a MAF having a range of from about 0.1 to 0.39, from about 0.1 to 0.38, from about 0.1 to 0.37, from about 0.1 to about 0.36, from about 0.1 to about 0.35, from about 0.1 to about 0.34, from about 0.1 to about 0.33, from about 0.1 to about 0.32, from about 0.1 to about 0.31, from about 0.1 to about 0.30, from about 0.1 to about 0.29, from about 0.1 to about 0.28, from about 0.1 to about 0.27, from about 0.1 to about 0.26, from about 0.1 to about 0.25, from about 0.1 to about 0.24, from about 0.1 to about 0.23, from about 0.1 to about 0.22, from about 0.1 to about 0.21, from about 0.1 to about 0.20, from about 0.1 to about 0.19, from about 0.1 to about 0.18, from about 0.1 to about 0.17, from about 0.1 to about 0.16, from about 0.1 to about 0.15, from about 0.1 to about 0.14, from about 0.1 to about 0.13, from about 0.1 to about 0.12, and/or from about 0.1 to about 0.11.

Nucleic acid targets may be of various concentrations in the reaction. The nucleic acid sample may be diluted or concentrated to achieve different concentrations of nucleic acids. The concentration of the nucleic acid targets in the nucleic acid sample may at least 1 genome copy equivalent per reaction, 2 genome copies equivalent per reaction, 5 genome copies equivalent per reaction, 10 genome copies equivalent per reaction, 20 genome copies equivalent per reaction, 30 genome copies equivalent per reaction, 40 genome copies equivalent per reaction, 50 genome copies equivalent per reaction, 100 genome copies equivalent per reaction, or more. In some cases, the concentration of the nucleic acids in the nucleic acid sample may be at most 1 genome copies equivalent per reaction, 2 genome copies equivalent per reaction, 3 genome copies equivalent per reaction, 5 genome copies equivalent per reaction, 10 genome copies equivalent per reaction, 20 genome copies equivalent per reaction, 40 genome copies equivalent per reaction, 50 genome copies equivalent per reaction, 100 genome copies equivalent per reaction, 1000 genome copies equivalent per reaction, 3000 genome copies equivalent per reaction, 5000 genome copies equivalent per reaction, 10000 genome copies equivalent per reaction or less.

### Nucleic Acid Enzymes

Mixtures and compositions of the present disclosure may comprise one or more nucleic acid enzymes. A nucleic acid enzyme may have exonuclease activity. A nucleic acid enzyme may have endonuclease activity. A nucleic acid enzyme may have RNase activity. A nucleic acid enzyme may be capable of degrading a nucleic acid comprising one or more ribonucleotide bases. A nucleic acid enzyme may be, for example, RNase H or RNase III. An RNase III may be, for example, Dicer. A nucleic acid may be an endonuclease I such as, for example, a T7 endonuclease I. A nucleic acid enzyme may be capable of degrading a nucleic acid comprising a non-natural nucleotide. A nucleic acid enzyme may be an endonuclease V such as, for example, an E. coli endonuclease V.

A nucleic acid enzyme may be a polymerase (e.g., a DNA polymerase). A DNA polymerase may be used. Any suitable DNA polymerase may be used, including commercially available DNA polymerases. A DNA polymerase refers to an enzyme that is capable of incorporating nucleotides to a strand of DNA in a template bound fashion. A polymerase may be Taq polymerase or a variant thereof.

Non-limiting examples of DNA polymerases include Taq polymerase, Tth polymerase, Tli polymerase, Pfu polymerase, VENT polymerase, DEEPVENT polymerase, EX-Taq polymerase, LA-Taq polymerase, Expand polymerases, Sso polymerase, Poc polymerase, Pab polymerase, Mth polymerase, Pho polymerase, ES4 polymerase, Tru polymerase, Tac polymerase, Tne polymerase, Tma polymerase, Tih polymerase, Tfi polymerase, Platinum Taq polymerases, Hi-Fi polymerase, Tbr polymerase, Tfl polymerase, Pfutubo polymerase, Pyrobest polymerase, Pwo polymerase, KOD polymerase, Bst polymerase, Sac polymerase, Klenow fragment, and variants, modified products and derivatives thereof. For certain Hot Start Polymerase, a denaturation step at 94° C.-95° C. for 2 minutes to 10 minutes may be required, which may change the thermal profile based on different polymerases. A nucleic acid enzyme may be capable, under appropriate conditions, of degrading an oligonucleotide probe. For example, a nucleic acid enzyme may be a polymerase and comprise exonuclease activity and degrade a probe resulting in a detectable signal. A nucleic acid enzyme may be capable, under appropriate conditions, of releasing a quencher from an oligonucleotide probe.

### Primers/Oligonucleotide Primers

In various aspects disclosed elsewhere herein, “primers” are used. Samples, mixtures, kits, and compositions of the present disclosure may comprise a primer, also referenced herein as an “oligonucleotide primer” or “amplification primer.” A primer of the present disclosure may be a deoxyribonucleic acid. A primer may be a ribonucleic acid. A primer may comprise one or more non-natural nucleotides. A non-natural nucleotide may be, for example, deoxyinosine. The oligonucleotide primer may be able to hybridize to a first analyte and a second analyte and may generates a first signal corresponding to said first analyte and a second signal corresponding to said second analytes.

A primer may comprise a first region complementary to an analyte and a second region comprising probe binding sites.

The analyte may be a patient derived oligonucleotide. The analyte may be a patient derived DNA. The analyte may be a patient derived RNA. The patient derived oligonucleotide may encode a sequence indicative of a organ or tissue transplant rejection described elsewhere herein.

The second region may comprise one or more than one probe binding sites. Each probe binding site encode a unique tag sequence. In certain embodiments, the number of unique probe binding sites in a primer is one or more than one. In certain embodiments, the number of unique probe binding sites in a primer is two or more than two. In certain embodiments, the number of unique probe binding sites in a primer is three or more than three. In certain embodiments, the number of unique probe binding sites in a primer is four or more than four. In certain embodiments the number of unique probe binding sites in a primer is between one to n_{pbs}. In certain embodiments, n_{pbs} is two or more than two. In certain embodiments, n_{pbs} is one, or 2, or 3, 4, 5, 6, 7, 8, 9, 10, 11, or 12. The probe binding sites may be the same or different compared to other primers in a reaction mixture. The primer may comprise combinations of probe binding sites that are different than the probe binding sites of other primers in a reaction mixture.

In certain embodiments, the number of unique tag sequence in a primer is one or more than one. In certain embodiments, the number of unique tag sequences in a primer is two or more than two. In certain embodiments, the number of unique tag sequences in a primer is three or more than three. In certain embodiments, the number of unique tag sequences in a primer is four or more than four. In certain embodiments the number of unique tag sequences in a primer is between one to nₜₛ. In certain embodiments, nₜₛ is two or more than two. In certain embodiments, nₜₛ is one, or 2, or 3, 4, 5, 6, 7, 8, 9, 10, 11, or 12. The probe binding sites may be the same or different compared to other primers in a reaction mixture. The primer may comprise combinations of probe binding sites that are different than the probe binding sites of other primers in a reaction mixture.

The primers may comprise additional regions that another primer can anneal to. For example, a primer can comprise a universal region that a universal primer can anneal to. A first reaction using a primer comprising a universal region can anneal to a target and generate, via extension or amplification, a nucleic acid that comprise the target nucleic acid sequence and the universal region. In a second reaction, the universal primer may anneal to the universal region and generate additional copies of the nucleic acid.

This may be especially advantageous for multiplexed workflows comprising multiple targets. For example, a mixture of different target specific primers that comprise universal regions can be used to amplify the multiple targets. As the resulting nucleic acids comprise universal regions, a universal primer can be used to amplify the multiple targets in a single reaction mixture, regardless of the original sequences of the targets.

As described elsewhere herein, amplification of targets can generate a signal via the degradation or removal of probes. Primers may comprise probe sites that may allow for targets to be labeled with probes sites via extension or amplification of the target specific primers. The probes may be allowed to anneal to the probe sites, and a second extension or amplification reaction may be performed to displace or degrade the probes, thereby generating a signal. This may be used in conjunction with primers with universal regions and the probe sites (and probes) may be three' to the universal region. The universal primers can then be used to generate the probe signal and can allow for multiplexed generation of signals from multiple targets.

A primer may comprise filler sequences. For example, a primer may comprise a sequence that does not anneal to a target, probe, or another primer. The filler sequence may have low or no binding to other sequences in the mixture. The filler sequence may be used to generate different primers that have a same or similar length that perform different functions. For example, a first primer may comprise two different probe binding sites and second primer may comprise one probe binding site and a filler sequence. The first primer may be able to bind two probes and generate two different signals, whereas the second primer may anneal to only one probe a generate one signal. Using the filler sequences the primers may be of comparable size may allow for improved multiplexing, for example, due to more similar melting temperatures or suitable reaction temperatures for the two primers.

A primer may comprise a blocking group or blocking region. The blocking group be at a three' end of an oligonucleotide. A blocking group may be unextendible and may need to be cleaved to allow a primer to be extended. The blocking group may allow for primers to differentiate between different loci or alleles, for example, those with single nucleotide polymorphisms (SNPs). For example, a blocking group may be unextendible and may need to be cleaved by an enzyme. The enzyme may recognize a perfectly matched primer-target duplex and may cleave the blocking group allowing for extension. A mismatched primer-target duplex may be unable to be recognized by the enzyme and fail to cleave off the blocking group, thereby blocking extension.

A primer may be a forward primer. A primer may be a reverse primer. In certain embodiments, the length of a primer may be between about five and about 150 nucleotides. In certain embodiments, the length of a primer may be at least 5, 10, 11, 12, 13, 14, 15, 16, 17, 18, 19, 20, 25, 30, 35, 40, 45, 50, 100, or 150 base pairs in length, or more. In certain embodiments, the length of a primer may be at most 150, 100, 50, 45, 40, 35, 30, 25, 20, 19, 18, 17, 16, 15, 14, 13, 12, 11, 10, or 5 nucleotides in length. In certain embodiments, the length of a primer may be about 5, 10, 11, 12, 13, 14, 15, 16, 17, 18, 19, 20, 25, 30, 35, 40, 45, 50, 75, 100, or 150 base pairs in length.Pl

A set of primers may comprise paired primers. Paired primers may comprise a forward primer and a reverse primer. A forward primer may be configured to hybridize to a first region (e.g., a 3′ end) of a nucleic acid sequence, and a reverse primer may be configured to hybridize to a second region (e.g., a 5′ end) of the nucleic acid sequence, thereby being configured to amplify the nucleic acid sequence under conditions sufficient for nucleic acid amplification. Different sets of primers may be configured to amplify different nucleic acid target sequences. For example, a first set of primers may be configured to amplify a first nucleic acid sequence of a given length, and a second set of primers may be configured to amplify a second nucleic acid sequence of shorter length than the first nucleic acid sequence. In another example, a first set of primers may be configured to amplify a first nucleic acid sequence of a given length, and a second set of primers may be configured to amplify a second nucleic acid sequence of longer length than the first nucleic acid sequence.

A mixture may comprise a plurality of forward primers and/or reverse primers. A plurality of forward primers and/or reverse primers may be a deoxyribonucleic acid. Alternatively, a plurality of forward primers and/or reverse primers may be a ribonucleic acid. A plurality of forward and/or reverse primers may be between about five and about 50 nucleotides in length. A plurality of forward and/or reverse primer may be at least 5, 10, 11, 12, 13, 14, 15, 16, 17, 18, 19, 20, 25, 30, 35, 40, 45, or 50 base pairs in length, or more. A plurality of forward primer and/or reverse may be at most 50, 45, 40, 35, 30, 25, 20, 19, 18, 17, 16, 15, 14, 13, 12, 11, 10, or 5 nucleotides in length.

A set of primers (e.g., a forward primer and a reverse primer) may be configured to amplify a nucleic acid sequence of a given length (e.g., may hybridize to regions of a nucleic acid sequence a given distance apart). The nucleic acid sequence may be encoded in a nucleic acid target. Aspects of nucleic acid sequence and nucleic acid target are disclosed elsewhere herein. A pair of primers may be configured to amplify a nucleic acid sequence of a length of at least 20, at least 30, at least 40, at least 50, at least 75, at least 100, at least 125, at least 150, at least 175, at least 200, at least 225, at least 250, at least 275, or at least 300 base pairs (bp), or more. A pair of primers may be configured to amplify a nucleic acid sequence of a length of at most 300, at most 275, at most 250, at most 225, at most 200, at most 175, at most 150, at most 125, at most 100, at most 75, or at most 50 bp, or less.

In some aspects, the primer may be configured to hybridize, anneal or be homologous to sequences derived from humans. In some aspects, a mixture may include one or more synthetic (or otherwise generated to be different from the target of interest) primers for PCR reactions.

In some aspects, a mixture may be subjected to conditions sufficient to anneal a primer to a nucleic acid molecule. In some aspects, a mixture may be subjected to conditions sufficient to anneal a plurality primer to a nucleic acid molecule.

In some aspects, a mixture may be subjected to conditions sufficient to anneal a plurality of primers to a plurality of nucleic acid targets. The mixture may be subjected to conditions which are sufficient to denature nucleic acid molecules. Subjecting a mixture to conditions sufficient to anneal an oligonucleotide primer to a nucleic acid target may comprise thermally cycling the mixture under reaction conditions appropriate to amplify the nucleic acid target(s) with, for example, polymerase chain reaction (PCR).

Conditions may be such that a primer pair (e.g., forward oligonucleotide primer and reverse oligonucleotide primer) are degraded by a nucleic acid enzyme. An oligonucleotide primer pair may be degraded by the exonuclease activity of a nucleic acid enzyme. A primer pair may be degraded by the RNase activity of a nucleic acid enzyme. Degradation of the primer pair may result in release of the primer. Once released, the primer pair may bind or anneal to a template nucleic acid molecule.

### Probes/Detection Probes/Oligonucleotide Probes

In various aspects disclosed elsewhere herein, “probes” are used. Samples, mixtures, kits, and compositions of the present disclosure may comprise a probe, also referenced herein as a “detection probe” or “oligonucleotide probe.” A probe may be a nucleic acid (e.g., DNA, RNA, etc.). A probe may comprise a region complementary to a region of a nucleic acid target. The nucleic acid target may be a nucleic acid encoding a sequence indicative of a organ or tissue transplant rejection in a subject. The concentration of a probe may be such that it is in excess relative to other components in a sample.

The probe may be able to hybridize to one or more corresponding analyte such that each probe-analyte complex would generate a corresponding signal.

A probe may comprise a non-target-hybridizing sequence. A non-target-hybridizing sequence may be a sequence which is not complementary to any region of a nucleic acid target sequence. A probe comprising a non-target-hybridizing sequence may be a hairpin detection probe. A probe comprising a non-target hybridizing sequence may be a molecular beacon probe. A probe comprising a non-target hybridizing sequence may be a molecular inversion probe. Examples of molecular beacon probes are provided in, for example, U.S. Pat. No. 7,671,184, incorporated herein by reference in its entirety. An probe comprising a non-target-hybridizing sequence may be a molecular torch. Examples of molecular torches are provided in, for example, U.S. Pat. No. 6,534,274, incorporated herein by reference in its entirety.

A sample may comprise more than one probe. Multiple probes may be the same or may be different. A probe may be at least lₚ nucleotides in length or at most lₚ in length. In some embodiments, lₚ can be 5 10, 11, 12, 13, 14, 15, 16, 17, 18, 19, 20, or more. In other embodiments, lₚ can be 2, 3, 4, 5, 6, 7, 8, 9, 10, 20, 30, 40, or 50. In some examples, a mixture comprises Np unique probes. In some cases, Np can be 2, 3, 4, 5, 6, 7, 8, 9, 10, 11, 12, 13, 14, 15, 20, 40, 60 or more. In other cases, Np can be 4, 8, 12, 24, 36, 48, 60.

As will be recognized and is described elsewhere herein, a probe may comprise a signal tag or a plurality of unique signal tags, which are described in more detail in other sections of this Application.

A probe may correspond to a region of a nucleic acid target. For example, a probe may have complementarity and/or homology to a region of a nucleic acid target. A probe may comprise a region which is complementary or homologous to a region of a nucleic acid target. In certain embodiments, a probe may have greater than 95% complementarity to a sequence of oligonucleotides on a nucleic acid target among a plurality of unique nucleic acid targets. In some embodiments, a probe may have less than 50%, 40%, 30%, 20%, 10%, 5%, or 1% complementarity to any member of a plurality of nucleic acid targets. A probe may have no complementarity to any member of the plurality of nucleic acid targets.

A probe corresponding to a region of a nucleic acid target may be capable of binding to the region of the nucleic acid target under appropriate conditions (e.g., temperature conditions, buffer conditions, etc.). For example, a probe may be capable of binding to a region of a nucleic acid target under conditions appropriate for polymerase chain reaction. A probe may correspond to an oligonucleotide which corresponds to a nucleic acid target. For example, an oligonucleotide may be a primer with a region complementary to a nucleic acid target and a region complementary to a probe.

A probe may be a molecular inversion probe or comprise a structure similar to a molecular inversion probe. For example, a probe may comprise (i) a first region at a first end of the probe that anneals to a nucleic acid target and (ii) a second region at a second end of the probe that anneals to the nucleic acid target at a different sequence. The oligonucleotide probe, when annealed to the target, may be able to be circularized via additional reactions, and generate a circularized probe. The oligonucleotide may be able to anneal to other probes (e.g., Taqman probes) and may comprise one or more probe binding sites. For example, an oligonucleotide may comprise from 5′ to 3′ (i) first region complementary to an analyte and a second region comprising probe binding sites, and a third region complementary to the analyte at different sequence. The second region may comprise more than one probe binding sites. The second region may comprise more than two probe binding sites. The second region may comprise more than three probe binding sites. The second region may comprise more four probe binding sites. The probe binding sites may be the same or different compared to other oligonucleotides in a reaction mixture. The oligonucleotide may comprise combinations of probe binding sites that are different than the probe binding sites of other oligonucleotides in a reaction mixture.

A probe may comprise filler sequences. For example, a probe may comprise a sequence that does not anneal to a target, primer, or another probe. The filler sequence may have low or no binding to other sequences in the mixture. The filler sequence may be used to generate different probes that have a same or similar length that perform different functions. For example, a first probe may comprise two different probe binding sites and second probe may comprise one probe binding site and a filler sequence. The first probe may be able to bind two probes and generate two different signals, whereas the second primer may anneal to only one probe a generate one signal. Using the filler sequences the probe may be of similar size may allow for improved multiplexing, for example, due to more similar melting temperatures or suitable reaction temperatures for the two probes.

A probe may comprise an uracil or other base that can be selectively recognized by an enzyme. For example, probe may comprise an uracil and may be cleaved via recognition of a Uracil-DNA glycosylases (UDG). For example, the probe may be circularized and then subsequently cleaved by a UDG.

The probe may be a universal probe. The probe may be non-specific to a specific analyte and bind to a region that is present in multiple different nucleic acids. As described throughout the disclosure, nucleic acids may be generated that have probe binding sites, such as via extension of a tailed primer or circularization of molecular inversion probe. These probe binding site may be universal probe binding sites, such that the sequence is common across different nucleic acids. Thus, the addition of a universal probe molecule can allow for binding to multiple different molecules and generating a signal from multiple molecules. For example, a FAM probe may have a set universal sequence. For a nucleic acid to generate a FAM signal, a sequence that binds to the FAM probe may be a part of the tail of the primer.

When encoding or barcoding analytes, the primers or probe may be designed to use the universal probe binding sequences to generate the signal associated with that probe.

A probe may be provided at a concentration Cₚ. In some cases, a second nucleic acid probe can be provided at a concentration of at least about nCₚ. In some cases, a second nucleic acid probe can be provided at a concentration of at most about nCₚ. In certain embodiments, n can be 2, 3, 4, 5, 6, 7, or 8.

In certain embodiments, n can be more than 8. Cₚ may be any concentration of a nucleic acid probe. In some cases, Cₚ is at least 1 nM, 5 nM, 10 nM, 20 nM, 30 nM, 40 nM, 50 nM, 100 nM, 150 nM, 200 nM, 250 nM, 300 nM, 350 nM, 400 nM, 450 nM, 500 nM, 1000 nM, or greater. In some cases, Cₚ is from about 1 nM to about 50 nM, from about 1 nM to about 30 nM, and/or from about 1 nM to about 25 nM. In some cases, Cₚ is at most 1000 nM, 500 nM, 450 nM, 400 nM, 350 nM, 300 nM, 250 nM, 200 nM, 150 nM, 100 nM, or 50 nM.

### Signal Tag

The signal tag also referred to herein as a “detectable agent” is capable of producing a fluorescent signal, electrochemical signal, chemiluminescent signal, and/or another quantifiable signal.

In some embodiments, the signal tag can comprise a “detectable label” can be a fluorescent label, such as a fluorophore, a fluorophore/quencher pair. A detectable label may be a chemiluminescent label. A “fluorophore” may be, for example, FAM, TET, HEX, TAMRA, ROX, JOE, Cy3, Cy5, Cy5.5, Cal Fluor Gold 540, Cal Fluor Orange 560, Cal Fluor Red 590, Cal Fluor Red 610, Cal Fluor Red 635, Quasar 570, Quasar 670, Quasar 705, ABY, Alexa Fluor 350, Alexa Fluor 405, Alexa Fluor 488, Alexa Fluor 532, Alexa Fluor 546, Alexa Fluor 555, Alexa Fluor 568, Alexa Fluor 594, Alexa Fluor 647, AlexaFluor 680, Alexa Fluor 750, ATTO 425, ATTO 550, ATTO 590, Cyan500, Texas Red, Fluorescein (FITC), BODIPY FL, Pacific Blue, Pacific Green, Coumarin, Oregon Green, Pacific Orange, VIC, LC610, CFR610, JA270, LC640, JUN, Trimethylrhodamine (TRITC), DAPI, APC, Cyan Fluorescent Protein (CFP), Green Fluorescent Protein (GFP), Red Fluorescent Protein (RFP), Phycoerythin (PE), quantum dots (for example, Qdot 525, Qdot 565, Qdot 605, Qdot 705, Qdot 800) or a derivative thereof, or an equivalent thereof. A fluorophore maybe FAM. A fluorophore may be HEX. A quencher may inhibit signal generation from a fluorophore. A “quencher” may be, for example, TAMRA, BHQ-1, BHQ-2, BHQ-3, Iowa Black, ZEN, Dabcy or an equivalent thereof. A quencher may be BHQ-1. A quencher may be BHQ-2. A quencher may be BHQ-3. In some embodiments, the signal tag can be a magnetic particle, and/or electrets structures exhibiting a permanent dipole.

Each probe used in the methods and assays of the presence disclosure may comprise at least one fluorophore. A fluorophore may be selected from any number of fluorophores. A set of fluorophores may be selected from 3, 4, 5, 6, 7, 8, 9, or 10 fluorophores, or more. One or more probes used in a single reaction may comprise the same fluorophore or the same set of fluorophores. In some cases, all probes used in a single reaction comprise the same fluorophore or the same set of fluorophores. Each probe may, when excited and contacted with its corresponding nucleic acid target, generate a signal. A signal may be a fluorescent signal. A plurality of signals may be generated from one or more probes.

Although fluorescent probes have been used to illustrate this principle, the disclosed methods are equally applicable to any other method providing a quantifiable signal, including an electrochemical signal, chemiluminescent signals, magnetic particles, and electrets structures exhibiting a permanent dipole.

In some cases, each probe in a mixture of a plurality of probes may comprise a same or similar signal tag. For example, a probe may comprise an identical signal tag to another probe. In some cases, a probe in a plurality of probes may comprise a different signal tag. In some cases, each probe comprises a different signal tag.

In some case, each fluorophore signal tag is capable of being detected in a single optical channel. In other case, a fluorophore may be detected in multiple channels. In some cases, a probe may have similar sequence or be capable or binding a similar sequence as another probe in the sample. In some cases, a probe may have a different sequence or be capable of binding a different sequence as compared to another probe in the sample.

### Signal Generation

Thermal cycling may be performed such that one or more probes are degraded by a nucleic acid enzyme. A probe maybe degraded by the exonuclease activity of a nucleic acid enzyme. A probe may generate a signal upon degradation. In some cases, a probe may generate a signal only if at least one member of a plurality of nucleic acid targets is present in a mixture.

In various aspects, extension reactions and amplification reactions may be used to allow for the generation of signals. The extension reaction and amplification reaction may be used to generate a signal correspond to an analyte. The extension reaction may extend an oligonucleotide that can hybridize to more than one analyte. Based on the hybridization partner, the extension reaction may generate a different signal. Extension or amplification of a first analytes may generate a first signal whereas the extension or amplification of a second analyte may generate a second signal. The efficiency of the hybridization reactions may affect the extension reaction or the generation of a signal. The extension or amplification reaction may generate a signal by degrading or reaction with the oligonucleotide that can hybridize to more than one analyte. The oligonucleotide that can hybridize to more than one analyte may be a probe, and the extension or amplification reaction may allow generation of a signal from the probe.

The probe may hybridize with different efficiency or affinity and may allow the generation of a different signal based on the analyte hybridized thereto.

Signal generation may correspond to reactions conditions of reactions relating to signal generation. The signal generation may be altered by a hybridization efficiency of the oligonucleotide.

For example, an oligonucleotide may have a hybridization efficiency to a first analyte and a different hybridization efficiency to a second analyte which may in turn affect the generation of signal or alter the resulting signal that is generated. In the case of amplification or extension reactions, a time period or temperature may be altered such to change the signal generation efficiency or a kinetic signature shape.

For example, a sample may comprise more than one analyte and an oligonucleotide that can hybridize to more than one analyte may be added to the sample. The different signal generation efficiency or kinetic shape of a reaction may be used to differentiate a first analyte and a second analyte. The annealing temperature of a reaction may be altered such that the hybridization to one analyte is favored over the hybridization to another analyte. Multiple reactions may be performed at different annealing temperature (for example using a gradient) that allows for a signal to be generated and distinguishable for different analytes. The reactions may be performed such that a first reaction has a more stringent annealing condition compared to a second reaction. The reactions may comprise an annealing time, and the annealing time may be modulated to affect the generation of a signal. For example, a first reaction may comprise an annealing time that is longer than an annealing time for a second reaction. For example, a first reaction may comprise an annealing time that is shorter than an annealing time for a second reaction.

Similarly, extension times and temperatures may be modulated to affect the generation of a signal and allow different signal to be obtained based on the analyte. For example, a first reaction may comprise an extension time that is longer than an extension time for a second reaction. For example, a first reaction may comprise an extension time that is shorter than an extension time for a second reaction. For example, a first reaction may comprise an extension temperature that is higher (or lower) than an extension temperature for a second reaction. For example, a first reaction may comprise an extension temperature that is lower than an extension temperature for a second reaction.

A reaction may generate one or more signals. A reaction may generate a cumulative intensity signal comprising a sum of multiple signals. A signal may be a chemiluminescent signal. A signal may be a fluorescent signal. A signal may be generated by a probe. For example, excitation of a probe comprising a luminescent signal tag may generate a signal. A signal may be generated by a fluorophore.

A fluorophore may generate a signal upon release from a hybridization probe. A reaction may comprise excitation of a fluorophore. A reaction may comprise signal detection. A reaction may comprise detecting emission from a fluorophore.

As will be recognized and is described elsewhere herein, a signal may be a fluorescent signal. A signal may correspond to a fluorescence intensity level. Each signal measured in the methods of the present disclosure may have a distinct fluorescence intensity value, thereby corresponding to the presence of a unique combination of nucleic acid targets. A signal may be generated by one or more probes.

Multiple signals may be generated by a probe. For example, an oligonucleotide may be able to bind to multiple analytes and may generate a signal corresponding to hybridization with a first analyte and a second signal corresponding with a second analyte.

S_{c} may be a number of signals detected in a single optical channel in an assay of the present disclosure. Sc may be at least 2, 3, 4, 5, 6, 7, 8, 9, 10, 11, 12, 13, 14, 15, 20, 25, 30, 40, 50 or more. Sc may be at most 50, 40, 30, 24, 20, 15, 14, 13, 12, 11, 10, 9, 8, 7, 6, 5, 4, 3, or 2. S_{c} may be 2, 3, 4, 5, 6, 7, 8, 9, 10, 11, 12, 13, 14, 15, 20, 25, 30, 40, or 50.

As will be recognized and is described elsewhere herein, sets of signals may be generated in multiple different optical channels, where each set of signals is detected in a single optical channel, thereby significantly increasing the number of nucleic acid targets that can be measured in a single reaction. In certain embodiments, S_{All} in may be the number of signals detected in all optical channel in an assay of the present disclosure. In certain embodiments, S_{All}=C×S_{c}, wherein C is the number of optical channels used to detect the signals. In some cases, two sets of signals are detected in a single reaction.

Each set of signals detected in a reaction may comprise the same number of signals, or different numbers of signals.

In some cases, a signal may be generated simultaneous with hybridization of an oligonucleotide probe to a region of a nucleic acid. For example, an oligonucleotide probe (e.g, a molecular beacon probe or molecular torch) may generate a signal (e.g., a fluorescent signal) following hybridization to a nucleic acid. In some cases, a signal may be generated subsequent to hybridization of an oligonucleotide probe to a region of a nucleic acid, following degradation of the oligonucleotide probe by a nucleic acid enzyme.

In cases where a probe comprises a signal tag, the probe may be degraded when bound to a region of a primer, thereby generating a signal. For example, a probe (e.g., a TaqMan® probe) may generate a signal following hybridization of the probe to a nucleic acid and subsequent degradation by a polymerase (e.g., during amplification, such as PCR amplification). A probe may be degraded by the exonuclease activity of a nucleic acid enzyme.

As will be recognized and is described elsewhere herein, a probe may comprise a quencher and a fluorophore, such that the quencher is released upon degradation of a probe, thereby generating a fluorescent signal. Thermal cycling may be used to generate one or more signals. Thermal cycling may generate at least 1, 2, 3, 4, 5, 6, 7, 8, 9, 10 signals, or more. Thermal cycling may generate at most 10, 9, 8, 7, 6, 5, 4, 3, 2, or 1 signal. Multiple signals may be of the same type or of different types. Signals of different types may be fluorescent signals with different fluorescent wavelengths. Signals of different types may be generated by detectable labels comprising different fluorophores. Signals of the same type maybe of different intensities (e.g., different intensities of the same fluorescent wavelength). Signals of the same type may be signals detectable in the same color channel. Signals of the same type may be generated by detectable labels comprising the same fluorophore. Detectable labels comprising the same fluorophore may generate different signals by nature of being at different concentrations, thereby generating different intensities of the same signal type.

In certain portions of this disclosure, the signal may be a fluorescent signal. For example, like fluorescent signals, any of the electromagnetic signals described above may also be characterized in terms of a wavelength, whereby the wavelength of a fluorescent signal may also be described in terms of color.

The color may be determined based on measuring intensity at a particular wavelength or range of wavelengths, for example by determining a distribution of fluorescent intensity at different wavelengths and/or by utilizing a band pass filter to determine the fluorescence intensity within a particular range of wavelengths.

The presence or absence of one or more signals may be detected. One signal may be detected, or multiple signals may be detected. Multiple signals may be detected simultaneously. Alternatively, multiple signals may be detected sequentially. A signal may be detected throughout the process of thermal cycling, for example, at the end of each thermal cycle. The signals may be detected in a multi-channel detector. For example, the signal may be observed using a detector that can observe a signal in multiple ranges of wavelengths simultaneously, substantially simultaneously, or sequentially. The signal may be observable in at least 1, 2, 3, 4, 5, 6, 7, 8, 9, 10, or more channels. The signal may be observable in no more than 10, 9, 8, 7, 6, 5, 4, 3, 2, or less channels.

In some cases, the signal intensity increases with each thermal cycle. The signal intensity may increase in a sigmoidal fashion. The presence of a signal may be correlated to the presence of at least one member of a plurality of target nucleic acids. Correlating the presence of a signal to the presence of at least one member of a plurality of target nucleic acids may comprise establishing a “signal intensity threshold.” A signal intensity threshold may be different for different signals. Correlating the presence of a signal to the presence of at least one member of a plurality of target nucleic acids may comprise determining whether the intensity of a signal increases beyond a signal intensity threshold. In some examples, the presence of a signal may be correlated with the presence of at least one of all members of a plurality of target nucleic acids. In other examples, the presence of a first signal may be correlated with the presence of at least one of a first subset of members of a plurality of target nucleic acids, and the presence of a second signal may be correlated with the presence of at least one of a second subset of members of a plurality of target nucleic acids.

The presence of a signal may be correlated to the presence of a nucleic acid target. The presence of least 1, 2, 3, 4, 5, 6, 7, 8, 9, 10, or more signals may be correlated with the presence of at least one of at least 1, 2, 3, 4, 5, 6, 7, 8, 9, 10, or more nucleic acid targets. The absence of a signal may be correlated with the absence of corresponding nucleic acid targets. The absence of least 1, 2, 3, 4, 5, 6, 7, 8, 9, 10, or more signals may be correlated with the absence of each of at least 1, 2, 3, 4, 5, 6, 7, 8, 9, 10, or more nucleic acid target molecules. The presence of a plurality of signals may be correlated with a combination of targets. The presence of a plurality of signals may be correlated with a unique combination of targets. For example, the detection of a particular plurality of signals may indicate the presence or absence of a unique or particular combination of targets.

### Digital Assays

In some aspects, the present disclosure provides methods for performing a digital assay. A method for performing a digital assay may comprise partitioning a plurality of nucleic acid targets and a plurality of probes into a plurality of partitions. In some cases, two, three, four, five, or more nucleic acid targets may be partitioned into a plurality of partitions together with two, three, four, five, six, seven, eight, nine, ten, 11, 12, 13, 14, 15, 16, 17, 18, 19, 20 or more probes. Following partitioning, the nucleic acid targets may be amplified in the partitions, for example, by polymerase chain reaction (PCR). Next, S signals may be generated from the oligonucleotide probes. Each signal of the S signals may correspond to the presence of a unique combination of nucleic acid targets in a partition. Following signal generation, the S signals may be detected in a single optical channel. The signals may be detected using, for example, fluorescence detection in a single-color channel.

A method for performing a digital assay may comprise amplifying nucleic acid targets derived from a sample in a plurality of partitions comprising probes complementary to one or more regions of nucleic acid targets. Each probe may be labeled with a fluorophore. The fluorophores may be capable of being detected in a single optical channel. For example, the fluorophores may each comprise similar emission wavelength spectra, such that they can be detected in a single optical channel. Following partitioning, S signals may be detected from the plurality of partitions if one or more of the nucleic acid targets is present. Each of the S signals may correspond to a unique combination of one or more of the nucleic acid targets present in a partition. From the S signals, the presence or absence of each of the nucleic acid targets in the sample may be determined.

At least one signal of the plurality of signals may correspond with the presence of a unique combination of two or more of the first or second pluralities of nucleic acid molecules in a single partition. For example, one signal may correspond to the presence of two nucleic acid molecules (e.g., two copies of a nucleic acid sequence) in a single droplet. A signal of the plurality of signals may correspond with two or more unique combinations of the first or second pluralities of nucleic acid molecules in a single partition (e.g., may be an ambiguous signal). For example, a signal may correspond with the presence of one nucleic acid molecule and may also correspond with the presence of two nucleic acid molecules.

A reaction may comprise generating a cumulative signal measurement. Assays of the present disclosure may comprise comparing two or more cumulative signal measurements to unambiguously detect any combination of nucleic acid targets in a sample. A cumulative signal measurement may comprise one or more signals generated from one or more probes provided to a sample solution. A cumulative signal measurement may be a signal intensity level which corresponds to the sum of signals generated from multiple oligonucleotide probes.

For example, two probes may each bind to different regions of the same nucleic acid molecule, where each probe generates a signal of a given wavelength at a S_{I}, the signal intensity level for the first probe binding to the nucleic acid molecule is S_{I1} and the signal intensity level for the first probe binding to the nucleic acid molecule is S_{I2}. Measurement of these signals would generate a cumulative signal measurement corresponding to the sum of both signal intensities (S_{I1}+S_{I2}). In some cases, S_{I1} equals S_{I2}, providing a cumulative signal measurement of 2S_{I1} or 2S_{I2}. In some embodiments, S_{I1} is different from S_{I2}. In certain embodiments, S_{I2}=nS_{I1}, where n is a number ≥1. In certain embodiments, n is a number selected from the list 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50. In certain embodiments, n is a number selected from the list 1, 2, 4, 8, 16, 32, 64, 128, 256, 512, and 1024. In certain embodiments, P is the number of unique probes that has binding specificity to a unique region on the same nucleic acid molecule. In certain embodiments, P is a number selected from the list 1, 2, 3, 4, 5, 6, 7, 8, 9, 10, and 11. In certain embodiments, each of P probes will generate a S_{IP}. In certain embodiments, the SI for each probe is selected from the list consisting of 2S_{I1}=S_{I2}, 4S_{I1}=S_{I3}, 8S_{I1}=S_{I4}, 16S_{I1}=S_{I5}, 32S_{I1}=S_{I6}, 64S_{I1}=S_{I7}, 128S_{I1}=S_{I8}, 256S_{I1}=S_{I9}, 512S_{I1}=S_{I10}, 1024S_{I1}=S_{I11}, and combinations thereof. In certain embodiments, the cumulative intensity S_{cum} is selected from the list of 1, 3, 7, 15, 31, 63, 127, 255, 511, 1023, and 2027.

As another example, two probes may each bind to a different nucleic acid molecule in a sample and/or a partition of a sample, where each probe generates a signal of a given wavelength at a S_{I}, and where the signal intensity level for the first probe binding to the nucleic acid molecule is S_{I1} and the signal intensity level for the first probe binding to the nucleic acid molecule is S_{I2}. Measurement of these signals would generate a cumulative signal measurement corresponding to the sum of both signal intensities (S_{I1}+S_{I2}). In some cases, S_{I1} equals S_{I2}, providing a cumulative signal measurement of 2S_{I1} or 2S_{I2}. In some embodiments, Sn is different from S_{I2}. In certain embodiments, S_{I2}=nS_{I1}, where n is a whole number ≥1. In certain embodiments, n is a number selected from the list 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50. In certain embodiments, n is a number selected from the list 1, 2, 4, 8, 16, 32, 64, 128, 256, 512, and 1024. In certain embodiments, P is the number of unique probes that is specific for an unique nucleic acid molecule in a sample. In certain embodiments, P is a number selected from the list 1, 2, 3, 4, 5, 6, 7, 8, 9, 10, and 11. In certain embodiments, each of P probes will generate a S_{IP}. In certain embodiments, the Sᵢ for each probe is selected from the list of 2S_{I1}=S_{I2}, 4S_{I1}=S_{I3}, 8S_{I1}=S_{I4}, 16S_{I1}=S_{I5}, 32S_{I1}=S_{I6}, 64S_{I1}=S_{I7}, 128S_{I1}=S_{I8}, 256S_{I1}=S_{I9}, 512S_{I1}=S_{I10}, 1024S_{I1}=S_{I11}, and combinations thereof. In certain embodiments, the cumulative intensity Sₘ is selected from the list consisting of 1, 3, 7, 15, 31, 63, 127, 255, 511, 1023, and 2027.

Methods of the present disclosure may comprise partitioning a sample or mixture into a plurality of partitions. A sample of mixture may comprise nucleic acids, oligonucleotide probes, and/or additional reagents into a plurality of partitions. A partition may be a droplet (e.g., a droplet in an emulsion). A partition may be a microdroplet. A partition may be a well. A partition may be a microwell. Partitioning may be performed using a microfluidic device. In some cases, partitioning is performed using a droplet generator. Partitioning may comprise dividing a sample or mixture into water-in-oil droplets. A droplet may comprise one or more nucleic acids. A droplet may comprise a single nucleic acid. A droplet may comprise two or more nucleic acids. A droplet may comprise no nucleic acids. Each droplet of a plurality of droplets may generate a signal. A plurality of signals may comprise the signal(s) generated from each of a plurality of droplets comprising a subset of a sample.

### Sample Processing

A sample may be processed concurrently with, prior to, or subsequent to the methods of the present disclosure. A sample may be processed to purify or enrich for nucleic acids (e.g., to purify nucleic acids from a plasma sample). A sample comprising nucleic acids may be processed to purity or enrich for nucleic acid of interest. A sample may undergo an extraction to extract molecules used in the assay. For example, the extraction may use a column to bind or interact with a molecule. For example, an RNA extraction kit may be used such as a Qiagen RNA mini kit to extract or isolate RNA. A sample may be diluted. A sample may be diluted at least at 1:1, 1:2, 1:3, 1:4, 1:5, 1:6, 1:7, 1:8, 1:9, 1:10, 1:11, 1:12, 1:13, 1:14, 1:15: 1:16: 1:17, 1:18, 1:19, 1:20, 1:30, 1:40, 1:50, 1:60, 1:70, 1:80, 1:90, 1:100, or 1:1000, 1:10000, 1:100000 or more. A sample may be diluted at no more than at 1:1, 1:2, 1:3, 1:4, 1:5, 1:6, 1:7, 1:8, 1:9, 1:10, 1:11, 1:12, 1:13, 1:14, 1:15: 1:16: 1:17, 1:18, 1:19, 1:20, 1:30, 1:40, 1:50, 1:60, 1:70, 1:80, 1:90, 1:100, or 1:1000, 1:10000, 1:100000, or less. The sample may be diluted in a buffer or a solution. For example, the sample may be diluted in Tris-Ethylenediaminetetraacetic acid (TE) buffer. The sample may be diluted with a solution comprising alcohol. The sample may be diluted with a solution comprising sodium acetate.

### Reactions

In various aspects disclosed elsewhere herein, reactions are performed. A reaction may comprise contacting nucleic acid targets with one or more probes. A reaction may comprise contacting a sample solution volume (e.g., a droplet, well, tube, etc.) with a plurality of oligonucleotide probes, each corresponding to one of a plurality of nucleic acid targets, to generate a plurality of signals generated from the plurality of oligonucleotide probes. A reaction may comprise polymerase chain reaction (PCR).

In some aspects, the methods may comprise circularization reactions. Molecular inversion probes, or oligonucleotides with similar structures may be used such that probes may present in a configuration, upon annealing to a target, that may allow for the two ends of the probes to be connected. For example, the two ends may be directly adjacent to one another, and a ligation reaction may join the two ends together to generate a circularized nucleic acid. The two ends may be more than one nucleotide away from each other and may be subjected to gap fill reactions, such as polymerization reactions, extension reactions, or other reactions that attach additional nucleotides to the ends of the oligonucleotides. The gap fill reactions may then be followed by a ligation reaction to circularize the nucleic acid.

In some aspects, the methods may comprise exonuclease, cleavage, or nucleic acid degradation reactions. An exonuclease may be used to selectively degrade certain nucleic acids. For example, probes may be added to a mixture and allowed to anneal to targets and the probes may be circularized. Probes that did not anneal to a target may remain linear. An exonuclease may be used to selectively remove the linear probes that did not bind to any target, leaving the probes that bound to targets intact. The remaining probes may be subjected to additional reactions such to generate signals associated with the target and allow for detection of targets. Similarly, enzymes may selectively cleave at a base type or sequence. For example, the probe may be circularized and subjected to a exonuclease reaction that degrades non-circularized oligonucleotides (e.g., probes that did not anneal to a target). The circularized probe may then be subjected to a cleavage reaction to linearize the circularized probe and allow for additional reaction to be performed on the probes. The cleavage reaction may use a restriction enzyme that recognizes specific sequences. A cleavage reaction may use an enzyme that recognizes a specific base, such as a uracil-DNA glycosylase, that cleaves at a uracil base.

In some aspects, the disclosed methods comprise nucleic acid amplification. Amplification conditions may comprise thermal cycling conditions, including temperature and length in time of each thermal cycle. The use of particular amplification conditions may serve to modify the signal intensity of a signal, thereby enabling a signal (or plurality of signals) to correspond to a unique combination of nucleic acid targets. Amplification may comprise using enzymes such to produce additional copies of a nucleic. The amplification reaction may comprise using oligonucleotide primers as described elsewhere herein. The oligonucleotide primers may use specific sequences to amplify a specific sequence. The oligonucleotide primers may amplify a specific sequence by hybridizing to a sequence upstream and downstream of the primers and result in amplifying the sequence inclusively between the upstream and downstream primer. The oligonucleotide may be able to amplify more than one sequence analyte by hybridizing upstream or downstream of multiple different sequences. The amplification reaction may comprise the use of nucleotide tri-phosphate reagents. The nucleotide tri-phosphate reagents may comprise using deoxyribo-nucleotide tri-phosphate (dNTPs). The nucleotide tri-phosphate reagents may be used as precursors to the amplified nucleic acids. The amplification reaction may comprise using oligonucleotide probes as described elsewhere herein. The amplification reaction may comprise using enzymes. Non-limiting examples of enzymes include thermostable enzymes, DNA polymerases, RNA polymerases, and reverse transcriptases. The amplification reaction may comprise generating nucleic acid molecules of a different nucleotide types. For example, a target nucleic acid may comprise DNA and an RNA molecule may be generated. In another example, an RNA molecule may be subjected to an amplification reaction and a cDNA molecule may be generated.

### Thermal Cycling

Methods of the present disclosure may comprise thermal cycling. Thermal cycling may comprise one or more thermal cycles. Thermally cycling may be performed under reaction conditions appropriate to amplify a template nucleic acid with PCR. Amplification of a template nucleic acid may require binding or annealing of oligonucleotide primer(s) to the template nucleic acid. Appropriate reaction conditions may include appropriate temperature conditions, appropriate buffer conditions, and the presence of appropriate reagents. Appropriate temperature conditions may, in some cases, be such that each thermal cycle is performed at a desired annealing temperature. A desired annealing temperature may be sufficient for annealing of an oligonucleotide probe(s) to a nucleic acid target. Appropriate buffer conditions may, in some cases, be such that the appropriate salts are present in a buffer used during thermal cycling Appropriate salts may include magnesium salts, potassium salts, ammonium salts. Appropriate buffer conditions may be such that the appropriate salts are present in appropriate concentrations. Appropriate reagents for amplification of each member of a plurality of nucleic acid targets with PCR may include deoxyribonucleotide triphosphates (dNTPs). dNTPs may comprise natural or non-natural dNTPs including, for example, dATP, dCTP, dGTP, dTTP, dUTP, and variants thereof.

In various aspects, primer extension reactions are utilized to generate amplified product. Primer extension reactions generally comprise a cycle of incubating a reaction mixture at a denaturation temperature for a denaturation duration and incubating a reaction mixture at an elongation temperature for an elongation duration. In any of the various aspects, multiple cycles of a primer extension reaction can be conducted. Any suitable number of cycles may be conducted. For example, the number of cycles conducted may be less than about 100, 90, 80, 70, 60, 50, 40, 30, 20, 10, or 5 cycles. The number of cycles conducted may depend upon, for example, the number of cycles (e.g., cycle threshold value (Ct)) used to obtain a detectable amplified product (e.g., a detectable amount of amplified DNA product that is indicative of the presence of a target DNA in a nucleic acid sample). For example, the number of cycles used to obtain a detectable amplified product (e.g., a detectable amount of DNA product that is indicative of the presence of a target DNA in a nucleic acid sample) may be less than about or about 100 cycles, 75 cycles, 70 cycles, 65 cycles, 60 cycles, 55 cycles, 50 cycles, 40 cycles, 35 cycles, 30 cycles, 25 cycles, 20 cycles, 15 cycles, 10 cycles, or 5 cycles. Moreover, in some embodiments, a detectable amount of an amplifiable product (e.g., a detectable amount of DNA product that is indicative of the presence of a target DNA in a nucleic acid sample) may be obtained at a cycle threshold value (Ct) of less than 100, 75, 70, 65, 60, 55, 50, 45, 40, 35, 30, 25, 20, 15, 10,or 5.

The time for which an amplification reaction yields a detectable amount of amplified nucleic acid may vary depending upon the nucleic acid sample, the sequence of the target nucleic acid, the sequence of the primers, the particular nucleic acid amplification reactions conducted, and the particular number of cycles of the amplification, the temperature of the reaction, the pH of the reaction. For example, amplification of a target nucleic acid may yield a detectable amount of product indicative to the presence of the target nucleic acid at time period of 120 minutes or less; 90 minutes or less; 60 minutes or less; 50 minutes or less; 45 minutes or less; 40 minutes or less; 35 minutes or less; 30 minutes or less; 25 minutes or less; 20 minutes or less; 15 minutes or less; 10 minutes or less; or 5 minutes or less.

In some embodiments, amplification of a nucleic acid may yield a detectable amount of amplified DNA at time period of 120 minutes or less; 90 minutes or less; 60 minutes; or less; 50 minutes or less; 45 minutes or less; 40 minutes or less; 35 minutes or less; 30 minutes or less; 25 minutes or less; 20 minutes or less; 15 minutes or less; 10 minutes or less; or 5 minutes or less.

### Accuracy, Specificity, and Sensitivity

The method of the disclosure may be able to correctly identify the presence of analyte in a sample and can be measured in terms of the accuracy of the assay, the sensitivity of the assay, the specificity of the assay.

Accuracy may be calculated as the total number of correctly classified samples divided by the total number of samples, e.g., in a test population. In some embodiments, the methods herein show an accuracy or rate of accuracy, R_{A}, for predicting the presence of an analyte. In certain embodiments, R_{A} is at least about 75% (e.g., R_{A} may be at least about: 75%, 80%, 85%, 90%, 95%, 97%, 99% or 100%). In certain embodiments, R_{A} is at least about 50% (e.g., R_{A} may be at least about: 55%, 60%, 65%, 70%, 75%, 80%, 85%, 90%, 95%, or more).

Specificity is a measure of the “true negatives” that are predicted by a test to be negative, and may be calculated as the number of correctly identified normal samples divided by the total number of normal samples. In certain embodiments, the methods herein show a specificity or rate of specificity, Rs, for predicting the presence of an analyte. In certain embodiments, Rs is at least about 75% (e.g., Rs may be at least about: 75%, 80%, 85%, 90%, 95%, 97%, 99% or 100%). In certain embodiments, Rs is at least about 50% (e.g., Rs may be at least about: 55%, 60%, 65%, 70%, 75%, 80%, 85%, 90%, 95%, or more).

Sensitivity is a measure of the “true positives” that are predicted by a test to be positive, and may be calculated as the number of correctly identified transplant rejection samples divided by the total number of transplant rejection samples. In certain embodiments, the methods herein show a specificity or rate of sensitivity, R_{N}, for predicting the presence of an analyte. In certain embodiments, R_{N} is at least about 75% (e.g., Rs may be at least about: 75%, 80%, 85%, 90%, 95%, 97%, 99% or 100%). In certain embodiments, R_{N} is at least about 50% (e.g., Rs may be at least about: 55%, 60%, 65%, 70%, 75%, 80%, 85%, 90%, 95%, or more).

### Kits

The present disclosure provides kits for sample collection. The kit may comprise a sample collection vessel or sample collection tube. The kit may comprise a sample collection tool or an object that can obtain a sample via the contact of cells or nucleic acids from the subject and transfer sample to a sample collection vessel or tube. The sample collection tool may comprise a swab.

The present disclosure also provides kits for performing assays or analysis of assay results. Kits may comprise one or more probes. Probes may be lyophilized. Different probes may be present at different concentrations in a kit. Probes may comprise a fluorophore and/or one or more quenchers.

Kits may comprise one or more sets of primers or probes as described herein. The kits may further comprise a set of primers comprising paired primers. Paired primers may comprise a forward primer and a reverse primer. A set of primers may be configured to amplify a nucleic acid sequence corresponding to particular nucleic acid target. For example, a forward primer may be configured to hybridize to a first region (e.g., a 3′ end) of a nucleic acid sequence, and a reverse primer may be configured to hybridize to a second region (e.g., a 5′ end) of the nucleic acid sequence, thereby being configured to amplify the nucleic acid sequence. Different sets of primers may be configured to amplify nucleic acid sequences. In one example, a first set of primers may be configured to amplify a first nucleic acid sequence, and a second set of primers may be configured to amplify a second nucleic acid sequence. Primers configured to amplify nucleic acid molecules may be used in performing the disclosed methods. In some cases, all of the primers in a kit are lyophilized.

As will be recognized and is described elsewhere herein, the nucleic acid target may comprise a sequence indicative of a tissue or organ transplant rejection. In certain embodiments, the sequence correlates with or is associated with a tissue or transplant rejection in a subject.

Kits may comprise one or more nucleic acid enzymes. A nucleic acid enzyme may be a nucleic acid polymerase. A nucleic acid polymerase may be a deoxyribonucleic acid polymerase (DNase). A DNase may be a Taq polymerase or variant thereof. A nucleic acid enzyme may be a ribonucleic acid polymerase (RNase). An RNase may be an RNase III. An RNase III may be Dicer. The nucleic acid enzyme may be an endonuclease. An endonuclease may be an endonuclease I. An endonuclease I may be a T7 endonuclease I. A nucleic acid enzyme may be capable of degrading a nucleic acid comprising a non-natural nucleotide. A nucleic acid enzyme may be an endonuclease V such as, for example, an E. coli endonuclease V. A nucleic acid enzyme may be a polymerase (e.g., a DNA polymerase). A polymerase may be Taq polymerase or a variant thereof. A nucleic acid enzyme may be capable, under appropriate conditions, of degrading an probe. A nucleic acid enzyme may be capable, under appropriate conditions, of releasing a quencher from an probe. Kits may comprise instructions for using any of the foregoing in the methods described herein.

### Systems

Methods as disclosed herein may be performed using a variety of systems. The systems may be configured such the steps of the method may be performed. For example, the systems may comprise a detector for the detection of signals as described elsewhere herein. The system may comprise a processor configured to process, receive, plot, or otherwise represent the data obtained from the detector. The processor may be configured to process the data as described elsewhere herein. The processor may be configured to generate a report of the results obtained from the assay. The results of the assay may be uploaded into a remote server, or other computer systems as described elsewhere herein. The results may be uploaded and sent to a subject's medical provider or an institution to detect, analyze, evaluate, screen for, prognose, diagnose, and/or monitor a tissue or organ transplant rejection.

The results obtained from the assay may be sent to the subject directly. The subject, medical provider, or other institution may be able to access the remote server such review or analyze the results. For example, the results may then be transmitted to another institution/or medical professional for monitoring or for providing recommendations for the subject. These results can then be uploaded into a cloud database or other remote database for storage and transmission to or access by a variety or individuals and institutions which may use the results of the assay. The results may be obtained on a smart phone or other computer system as disclosed elsewhere herein which may display the results.

The present disclosure provides computer systems that are programmed to implement methods of the disclosure. The computer system can perform various aspects of the present disclosure. The computer system can be an electronic device of a user or a computer system that is remotely located with respect to the electronic device. The electronic device can be a mobile electronic device.

The computer system may include a central processing unit (CPU, also “processor’ and “computer processor” herein), which can be a single core or multi core processor, or a plurality of processors for parallel processing. The computer system may include memory or memory location (e.g., random-access memory, read-only memory, flash memory), electronic storage unit (e.g., hard disk), communication interface (e.g., network adapter) for communicating with one or more other systems, and peripheral devices, such as cache, other memory, data storage and/or electronic display adapters. The memory, storage unit, interface and peripheral devices are in communication with the CPU through a communication bus (solid lines), such as a motherboard. The storage unit can be a data storage unit (or data repository) for storing data. The computer system can be operatively coupled to a computer network (“network”) with the aid of the communication interface. The network can be the Internet, an internet and/or extranet, or an intranet and/or extranet that is in communication with the Internet. The network in some cases is a telecommunication and/or data network. The network can include one or more computer servers, which can enable distributed computing, such as cloud computing. The network, in some cases with the aid of the computer system, can implement a peer-to-peer network, which may enable devices coupled to the computer system to behave as a client or a server.

The CPU can execute a sequence of machine-readable instructions, which can be embodied in a program or software. The instructions may be stored in a memory location, such as the memory. The instructions can be directed to the CPU, which can subsequently program or otherwise configure the CPU to implement methods of the present disclosure. Examples of operations performed by the CPU can include fetch, decode, execute, and writeback.

The CPU can be part of a circuit, such as an integrated circuit. One or more other components of the system can be included in the circuit. In some cases, the circuit is an application specific integrated circuit (ASIC).

The storage unit can store files, such as drivers, libraries and saved programs. The storage unit can store user data, e.g., user preferences and user programs, or raw data or processed results from the assays. The computer system in some cases can include one or more additional data storage units that are external to the computer system, such as located on a remote server that is in communication with the computer system through an intranet or the Internet.

The computer system can communicate with one or more remote computer systems through the network. For instance, the computer system can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system via the network.

Methods as described herein can be implemented by way of machine (e.g., computer processor) executable code stored on an electronic storage location of the computer system, such as, for example, on the memory or electronic storage unit. The machine executable or machine-readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processor adapted to execute the code, or can be compiled during runtime. The code can be supplied in a programming language that can be selected to enable the code to execute in a precompiled or as-compiled fashion.

Aspects of the systems and methods provided herein, such as the computer system, can be embodied in programming. Various aspects of the technology may be thought of as “products” or “articles of manufacture” typically in the form of machine (or processor) executable code and/or associated data that is carried on or embodied in a type of machine readable medium. Machine executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, f or example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system can include or be in communication with an electronic display that comprises a user interface (UI) for providing, for example, plots of data, plots of kinetic signatures, information relating to signal amplitude, Examples of UIs include, without limitation, a graphical user interface (GUI) and web-based user interface.

Methods and systems of the present disclosure can be implemented by way of one or more algorithms. An algorithm can be implemented by way of software upon execution by the central processing unit. The algorithm can, for example, parameterize data points or fit data point to specified mathematical functions, in order to quantify analytes.

### EXAMPLES

### Example 1

### Whole Blood Collection

A whole blood sample is collected from a transplant recipient (i.e. a patient having received one or more organ or tissue from a donor). Exclusion criteria include transplant patients receiving a transplant from their monozygotic twin. The organ donors who are pregnant or are diagnosed for cancer are also excluded. Blood collection is a critical step and can form the basis for the method. Thus, it is important to properly collect, label, store and transport the sample, to minimize contamination. It is also important to ensure that safe and best practices for phlebotomy are followed and all patient consents are collected.

Standard blood collection methods can be used. A preferred method for blood collection is venipuncture, where a needle is inserted into a vein, usually in the arm, and blood is collected into a sterile tube. The tube can be either a standard EDTA tube or a tube specifically to preserve cell free DNA (i.e. Streck tube). The collected blood sample is properly labeled, stored, and transported to the laboratory for further processing.

Alternatively, blood can be collected using other methods such as a finger prick or from a central line, if available. It is important to draw as much blood as possible into the tube (i.e., up to 10 mL) so the chemicals in the tubes are at the correct concentrations. The tubes containing the blood can be stored at room temperature until plasma separation. It is important to note the volume of blood needed, the recipient's condition, and any other factors that may affect the quality or quantity of the collected blood sample.

### Example 2

### Plasma Separation

It is also important to properly isolate the plasma from the blood collected in Example 1 and ensure the isolated plasma has minimal contamination and degradation. For example, care should be taken to ensure that the plasma is properly isolated and free of any contamination from other blood components. Plasma can be separated from the blood collected in Example 1 using centrifugation. For example, the blood sample can be centrifuged at the specified conditions to separate the blood components based on their density. A double centrifugation process can also be used. In this double centrifugation process, the whole blood is centrifuged in a first round, producing a supernatant containing the plasma. The plasma layer, which contains the cell free DNA (cfDNA) can be carefully pipetted off such that the other blood components are not disturbed. A second round of centrifugation can be performed on the plasma to remove any remaining cells or cellular components.

Alternatively, plasma separation can be done using other methods such as filtration or using a specialized device designed for plasma separation. It is important to minimize genomic DNA contamination, which can reduce the sensitivity of the assay. In general, if the blood is drawn into a cell free DNA tube (i.e. Streck), the cfDNA is stable in the whole blood for up to 2 weeks. If the blood is drawn into an EDTA tube, the cfDNA is stable in the whole blood for up to 4 hours. It is essential to perform the plasma separation within these time periods to minimize genomic DNA contamination and/or degradation.

Following centrifugation, the plasma can be stored at less or equal to −20° C. to ensure DNA stability.

### Example 3

### Isolating cfDNA from Plasma

It is important to properly isolate the cfDNA from the plasma isolated in Example 2 and ensure that the cfDNA is free of any contamination. cfDNA is fragmented DNA that is circulating freely in the bloodstream and is often derived from cell death. cfDNA can originate from the host or the donor organ, and its analysis can provide valuable information about the transplant status.

For example, cfDNA can be isolated from the plasma using commercial kits and following the manufacturer's instructions. Examples of preferred commercial kits include the Qiagen cfDNA kit, Qiasymphony, Qiagen EZ1/EZ2, Roche MagnaPure, and Thermo Fisher MagMax cfDNA kit.

Alternatively, cfDNA can be isolated using other methods such as phenol-chloroform extraction or magnetic bead-based extraction. The cfDNA can be eluted into a stabilizing buffer, including Tris-EDTA (TE) buffer or water. The DNA must be isolated such that it is free of any contamination, including but not limited to, PCR inhibitors, proteins, exogenous DNA, and other chemicals which can degrade DNA (i.e. strong acids/bases).

### Example 4

### Enriching Select Regions

It is important to ensure that a selected regions of the genome are properly enriched and free of any contamination.

To enrich the genome from the isolated cfDNA of Example 3, from about 24 to about 384 regions of interest are enriched. Enrichment can be done in a single volume of reaction. Enrichment can be done in two or more volumes of reactions. These regions are selected based on the presence of a SNP within the region, and the enrichment regions can be fixed for all recipients. There is no a priori knowledge of donor or host SNP genotypes. The preferred method for enriching select regions of the genome is by using target enrichment techniques such as ligation, hybridization capture, or PCR amplification. This step is crucial for the success of the method as it ensures that the SNPs of interest are present in sufficient quantities for detection in the subsequent digital PCR step. In some cases, a single copy from a given target region can be sufficient. Alternatively, other methods for enriching select regions of the genome can be used, such as molecular inversion probes or selective circularization. Such methods are developed to minimize amplification bias. It is crucial that all targets amplify with similar efficiencies such that downstream analysis is not biased. Additionally, the amplification must be performed in a controlled manner to prevent cross-contamination or prior amplicon contamination. The product of the amplification will be used in digital PCR across one or greater wells.

### Example 5

### Performing Digital PCR Multiplex SNP Detection Reactions

Once the selected regions of the genome have been enriched, the next step is to perform a set of digital PCR multiplex SNP detection reactions. For an eight reaction multiplex assay, a minimum 10 ng of nucleic acid (corresponding to approximately 3000 copies) are used in the enrichment reactions to generate a minimum of 40,000 copies of the nucleic acids, which are split into the eight reaction assays. Digital PCR is a highly sensitive and accurate method for quantifying nucleic acids. It involves amplifying, in a single volume of reaction containing a master mix and a sample containing a plurality of analytes, such that thousands or tens of thousands of partitions within the single volume each containing a single analyte molecule or no analyte molecules, and then the single volume of reaction is cycled through a PCR reaction such that an analyte, if present in a droplet, can be amplified in the PCR reaction. At the end of the PCR, each partition will either contain amplified analyte (positive) or no amplified analyte (negative), and the number of positive partitions can be used to accurately quantify the amount of target analyte in the sample.

In this method, the digital PCR is highly multiplexed to detect many SNPs simultaneously. The product of the enrichment reaction is split across multiple detection reactions, with the same detection reactions used for all recipient samples. Each detection reaction detects 12 target alleles using 6 channels and a calibrator target. However, the method can detect at least N+1 SNP nucleic acid sequences, where N is the number of color channels on the detection instrument. The detected nucleic acid sequences may comprise one or more of the multiple possible nucleic acid sequences for a given SNP. This step is crucial for the success of the method as it ensures that the SNPs of interest are accurately detected and quantified.

Alternatively, other methods for SNP detection can be used, such as allele-specific PCR or high-resolution melting analysis. It is important to ensure that the digital PCR is properly performed and that the results are accurately analyzed.

### Example 6

### Analyzing Donor Informative SNPs to Determine Total Level of Donor-Derived cfDNA

After performing the digital PCR multiplex SNP detection reactions, it is important to ensure the analysis of the donor informative SNPs counts across all reactions is properly performed and the total level of donor-derived cfDNA present in the same are accurately interpreted. Donor informative SNPs are identified based on the relative copy number of each target relative to all other targets. The donor informative SNPs counts across all reactions are analyzed together to determine the total level of donor-derived cfDNA present. This step is crucial for the success of the method as it ensures that the total level of donor-derived cfDNA is accurately determined. This information can then be used to determine the rejection probability. Alternatively, other methods for analyzing donor informative SNPs can be used, such as next-generation sequencing or microarray analysis.

### Example 7

### Determining Rejection Probability

Rejection probability is determined based on the measured donor-derived cfDNA copy number, the ratio of donor-derived cfDNA copy number to the measured recipient cfDNA copy number, and thresholds set based on a clinical study where biopsy comparator results are known.

### Example 8

### Determining Ideal SNP Targets and Population Level Presence

In detection methods targeting only the “rarer allele” of a bi-allelic SNP, a population minor allele frequency from about 0.1 to about 0.4 can provide a higher proportion of informative loci per marker than detecting both alleles. For a marker to be informative in detecting donor derived cfDNA the marker must be present in the donor and absent in the recipient. The optimal allele frequency of the detected allele is 1−\sqrt{0.5}. This give the highest proportion of informative loci. In unrelated donor recipient pairs the recipient will be homozygous for the undetected allele 50% of the time and the donor will have the detected allele 50% of the time leading to 0.25 probability of a loci being informative while only using 1 distinct marker address. When selecting SNPs, target patient population genetic backgrounds can be considered to optimize the assay. Particular considerations are made to optimize primer and probe design, including thermodynamics, dimer formation, sequence context and compatibility with other primers.

In a method utilizing the detection of both alleles of a bi-allelic SNP the optimal frequency of each allele is 0.5 optimizing the recipient being a homozygote for either allele 50% of the time and an unrelated donor having at least 1 copy of the alternative allele 75% of the time leading to a 0.375 probability of a loci being informative but using 2 distinct marker addresses.

TABLE 3
Informative Loci Functions

| | Rare Allele Detection | | | Both Allele detection | | |
|---|---|---|---|---|---|---|
| Relationship | Informative Loci | p = 0.5 | p = 0.71 | Informative Loci | p = 0.5 | p = 0.71 |
| Unrelated | p² − p⁴ | 0.188 | 0.25 | p² − p⁴ + q² − q⁴ | 0.375 | 0.328 |
| Full-sibling | 0.1875 * (2pq)² + p³q | 0.109 | 0.136 | 0.375 * (2pq)² + p³q + q³p | 0.219 | 0.186 |
| Half-sibling | p³q + 0.5 * p²q(1 + q) | 0.156 | 0.198 | p³q + 0.5 * p²q(1 + [symbol] | 0.313 | 0.268 |
| Parent-child | p²q | 0.125 | 0.146 | pq(p + q) | 0.25 | 0.207 |

[symbol]

indicates data missing or illegible when filed

### Example 9

### Single SNP Per Target Allele

It is important to maximize the information obtained from each multiplex dPCR reaction and increase the efficiency of the method. A preferred method may involve analyzing only one allele at each chosen SNP site. While dPCR is a highly sensitive and precise technique that allows the quantification of specific nucleic acid sequences in a sample, its capacity is limited by the level of multiplexing on the detection instrument. Since a single SNP site can have multiple alleles, a comprehensive analysis of all of the alleles at the SNP site would require two, three, or four markers, depending on the number of possible alleles at the SNP site. This would significantly reduce the number of SNP sites that could be analyzed simultaneously.

For example, if the detection instrument has four color channels with no within-channel multiplexing capability, and there are four possible alleles at a single SNP site, then analyzing all alleles at that site would utilize all four channels, leaving no additional channel available for analyzing other SNP sites. In a preferred method of by analyzing only one allele at each chosen SNP site, it is possible to analyze four different SNP sites simultaneously, one for each channel. This increases the overall number of SNP sites that can be analyzed in a single reaction, thereby increasing the efficiency of the method.

Therefore, it is sometimes more efficient to read out one allele at more sites than more alleles at fewer sites. This approach allows for a more comprehensive and accurate assessment of the transplant status without the need for a priori knowledge of donor or host SNP genotypes. This is also important in transplant rejection monitoring, where it is crucial to analyze a broad range of SNPs to accurately determine the total level of donor-derived cfDNA and the rejection probability. Ultimately, this approach maximizes the information obtained from each reaction and improves the overall efficiency of the method.

### Example 10

### Analysis of Preamplification Bias

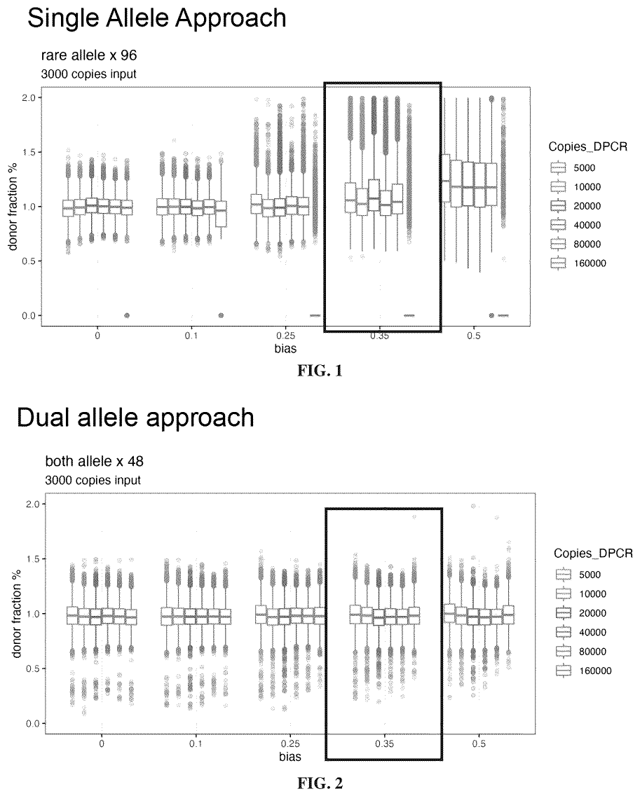

Pre-amplification biases are simulated. An expected fraction of 1.0% interrogating both a single allele per loci and both alleles per loci are assumed. Distributions of observed donor fraction are calculated across each bias for multiple DNA copy inputs into digital PCR. Within an expected bias range, the distribution of observed donor fractions remains similar when interrogating both alleles (see FIG. 1 and FIG. 2). In the single allele approach, it would be difficult to discriminate donor allele fractions ranging from 0.5%-2% (see FIG. 1), whereas the pre-amplification bias does not affect the ability to precisely determine allele fraction in the dual allele approach. Thus, it is determined that a dual allele approach is preferred over a single allele approach.

### Example 11

### Test for Inclusivity and Population Allele Frequency

In a dual allele approach, we want to determine the inclusivity for interrogating both alleles of a minimum of 48 loci. Simulations are completed having an assumed population allele frequency average of 0.35, to account for the range of minor allele frequencies from 0.3-0.4 for all major population groups in 1,000 genomes. An overall inclusivity >99% of the population with 4 informative loci can be derived from a panel of 48 loci (see Table 1).

| | Average allele | Min. infor- | | |
|---|---|---|---|---|
| # loci | frequency | mative loci | Relationship | Inclusivity |
| 48 | 0.35 | 4 | Unrelated | 1 |
| 48 | 0.35 | 4 | Full-sibling | 0.9917 |
| 48 | 0.35 | 4 | Parent-child | 0.9979 |
| 48 | 0.35 | 4 | Half-sibling | 0.9999 |
| 72 | 0.35 | 4 | Unrelated | 1 |
| 72 | 0.35 | 4 | Full-sibling | 0.9997 |
| 72 | 0.35 | 4 | Parent-child | 1 |
| 72 | 0.35 | 4 | Half-sibling | 1 |
| 96 | 0.35 | 4 | Unrelated | 1 |
| 96 | 0.35 | 4 | Full-sibling | 1 |
| 96 | 0.35 | 4 | Parent-child | 1 |
| 96 | 0.35 | 4 | Half-sibling | 1 |

### Example 12

### In Silico Analysis

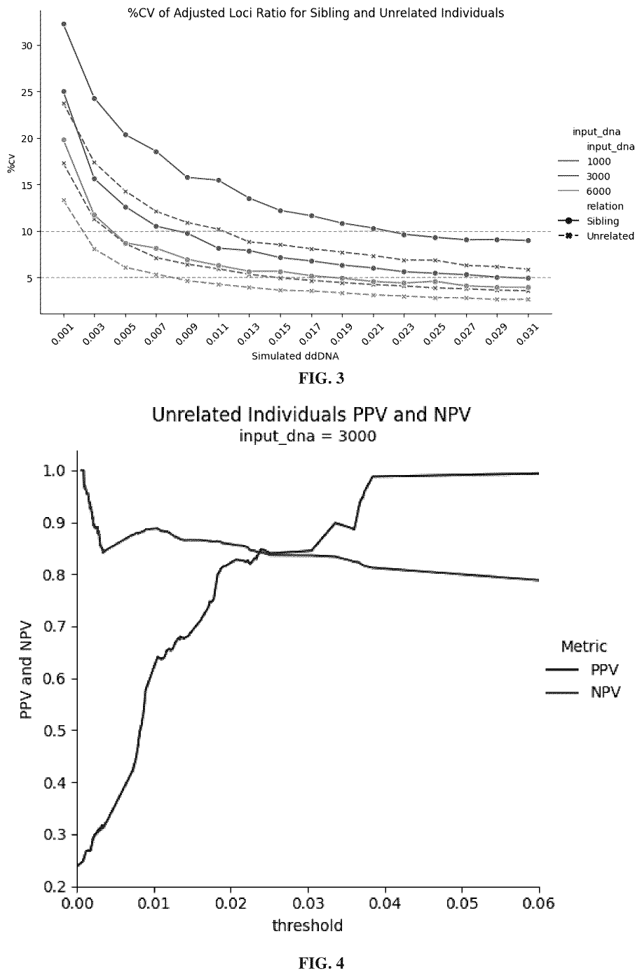

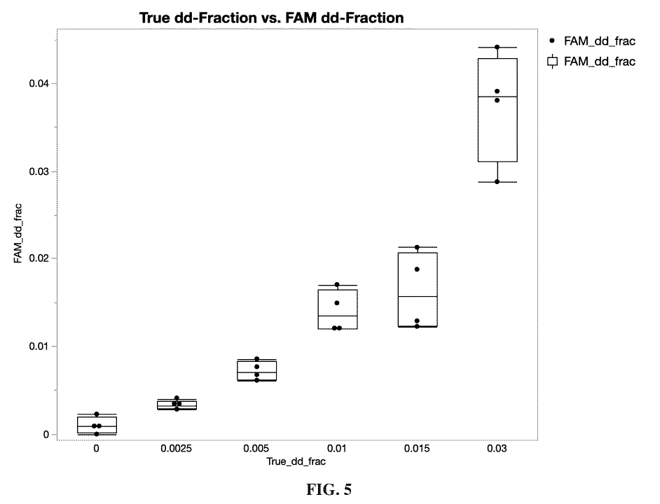

Simulations are conducted to understand the variability at each donor allele fraction with the assumption of 48 loci (96 alleles). Noise is simulated at various components of the assay, including extraction noise, pre-amplification bias, dilution noise, dPCR noise, and Poisson noise. The simulation shows that with 3,000 copies of template DNA (equivalent to approximately 10 ng human genomic DNA), the percent coefficient of variation (% CV) is approximately 6% at a 1% donor fraction (see FIG. 3, which illustrates the % CV as a function of donor fraction for various DNA input copies into pre-amplification and relationship status). This illustrates the potential of a 48 loci assay on digital PCR.

Additionally, we simulate the positive and negative predictive values for the 48 loci assay assuming 3,000 DNA copy inputs. FIG. 4 demonstrates at 1% donor fraction a positive predictive value (PPV) of approximately 65% with an negative predicative value (NPV) of approximately 90%, further proving the potential of an assay to mitigate the need for biopsies.

### Example 13

### Single Well Assay

A single well is built, which includes a calibrator in every channel. This calibrator is used as a reference in the calling algorithm to determine the location of each target based on the differences in fluorescent intensity. Each allele is located at either 2 or 4 times the fluorescent intensity of the calibrator in a given channel.

We perform studies using contrived samples for each loci of a single well, as well as comprehensive analysis of a full well. For each loci, the following is tested prior to exclude pre-amplification:

| Factor | Level | Levels |
|---|---|---|
| Panels | 2 | Cell line sheared to ~160 bp via covaris. Analysis |
| | | on a Bioanalyzer can ensure >95% of DNA are |
| | | less than 500 bp. The distributions of size are |
| | | approximately equal based on PCR assays. Donor |
| | | cell line contains unique marks from recipient |
| | | cell line |
| | | Panel 1 = Ref. allele information |
| | | Panel 2 = Alt. allele information |
| DNA input | 2 | 5000 copies (donor + recipient) to represent a low |
| amount into | | end input |
| dPCR | | These will be quantified using results of the |
| | | screening studies and correlated with Qubit. |
| dd-cfDNA | 12 | Panels 1 and 2 only. |
| amount | | Panel 1 assumes donor has alt allele. Panel 2 |
| | | assumes donor has ref allele. Ref allele |
| | | informative 100%, 3%, 1.5%, 1%, 0.5%, 0.25% |
| Replicates per | 4 | 96 per SNP |
| condition | | |

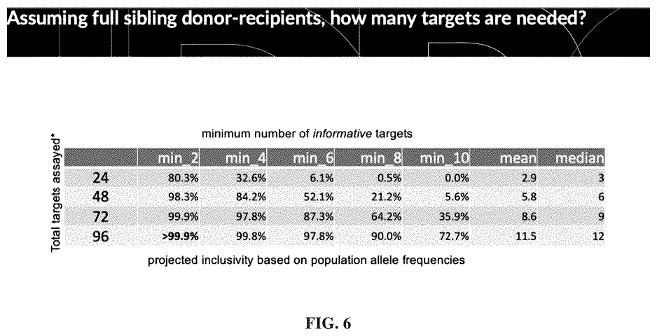

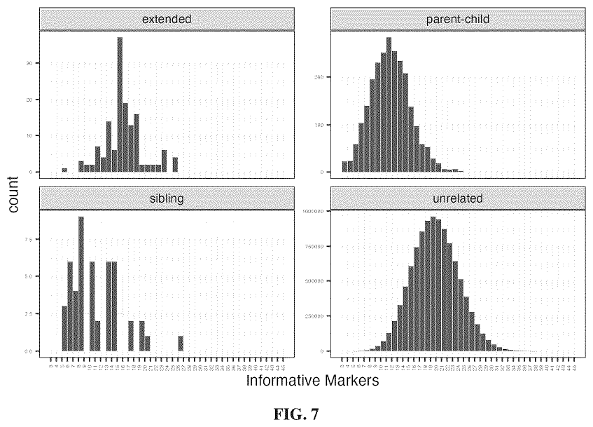

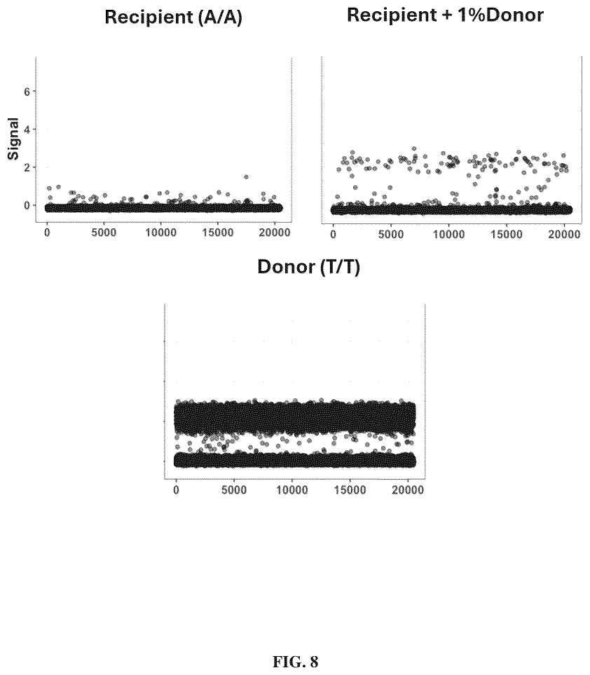

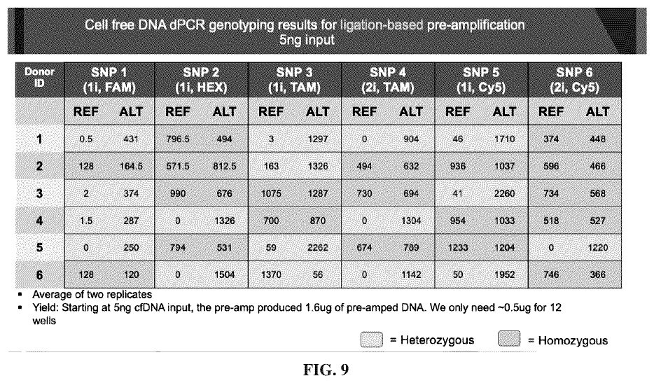

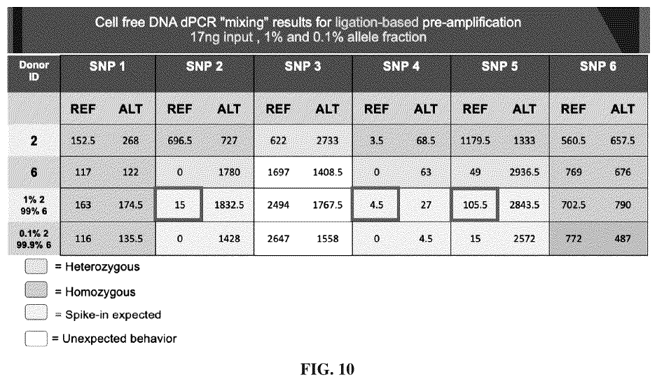

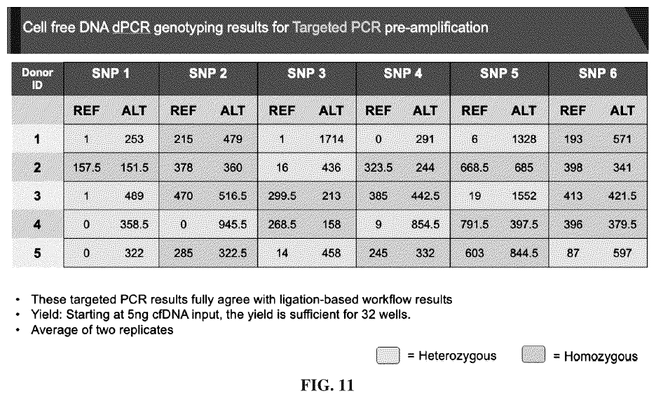

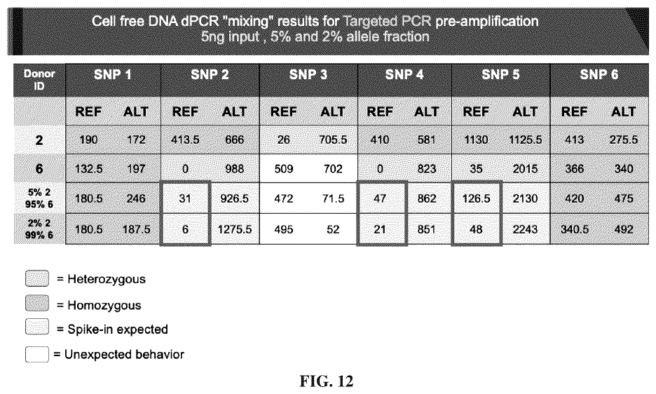

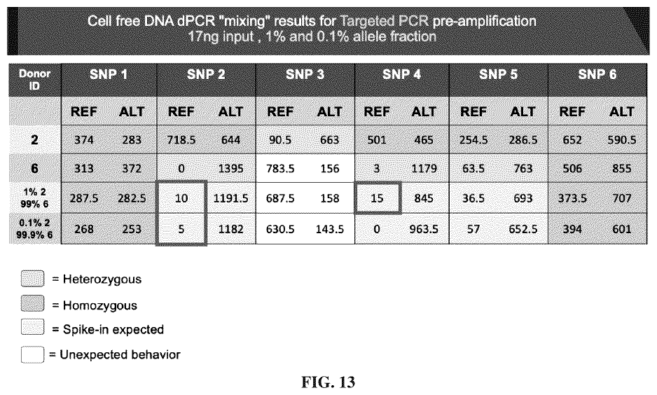

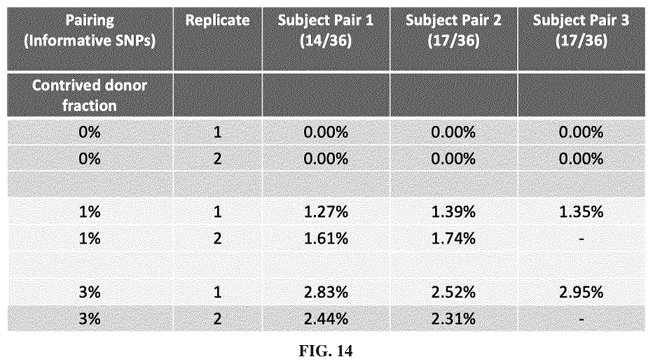

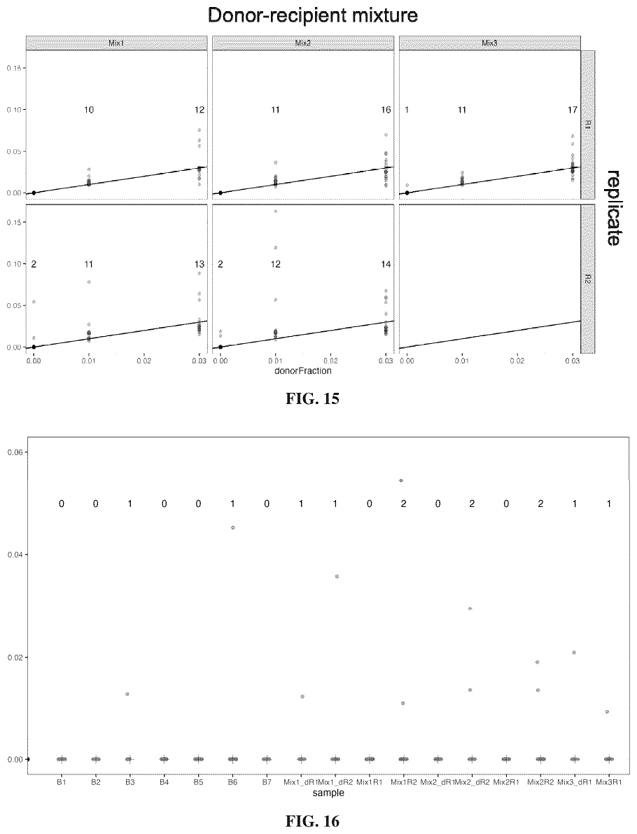

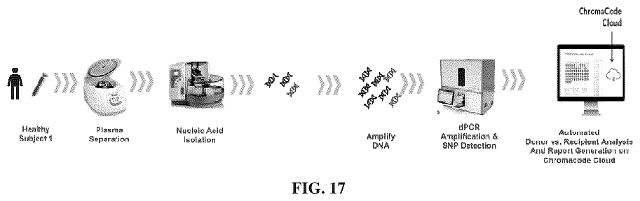

2 SNPs (i.e. 4 targets) are leveled such that there are 2 reference alleles in FAM, and 2 alternate alleles in HEX. Each allele is at a unique fluorescent intensity level and can be uniquely distinguished by the calling algorithm. Contrived spike ins are made such that the alternate alleles are associated with recipients, and reference alleles are associated with donors. Thus, one would expect to see an increase in signal in FAM as the donor fraction increase. FIG. 6 demonstrates this for a single allele. This process can be scaled up to account for all 96 alleles across 8 wells.

### REFERENCES

The following publications, references, patents and patent applications are hereby incorporated by reference in their entireties.
Schwarz, A et al., Safety and Adequacy of Renal Transplant Protocol Biopsies, American Journal of Transplantation, 2005; 5(8):992-1996.
Limaye, A R et al., Liver biopsy after liver transplantation. 2012. Available at http://www.intechopen.com/books/liver-biopsy-indicationsprocedures-results/liver-biopsy-after-liver-transplantation.
Eikmans, M et al., Non-invasive Biomarkers of Acute Rejection in Kidney Transplantation: Novel Targets and Strategies. Front Med (Lausanne). 2019; 5:358.
Clausen, F B et al., Droplet digital PCR-based testing for donor-derived cell-free DNA in transplanted patients as noninvasive marker of allograft health: Methodological aspects. PLoS One. 2023; 8(2), p.e0282332.
Beck, J et al., Digital Droplet PCR for Rapid Quantification of Donor DNA in the Circulation of Transplant Recipients as a Potential Universal Biomarker of Graft Injury. Clinical Chemistry. 2013; 59:1732-41.
Beck, J et al. Donor-Derived Cell-Free DNA Is a Novel Universal Biomarker for Allograft Rejection in Solid Organ Transplantation. Transplantation Proceedings. 2015; 47:2400-03.
Beck, J et al., A Universal Droplet Digital PCR Approach for Monitoring of Graft Health After Transplantation Using a Preselected SNP Set. Methods in Molecular Biology. 2018; 1768:335-348.
Schütz E, Fischer A, Beck J, Harden M, Koch M, Wuensch T, Stockmann M, Nashan B, Kollmar O, Matthaei J, Kanzow P, Walson P D, Brockmöller J, Oellerich M. Graft-derived cell-free DNA, a noninvasive early rejection and graft damage marker in liver transplantation: A prospective, observational, multicenter cohort study. PLoS Med. 2017 Apr. 25; 14(4):e1002286. doi: 10.1371/journal.pmed.1002286. PMID: 28441386; PMCID: PMC5404754.
Oellerich M, Sherwood K, Keown P, Schitz E, Beck J, Stegbauer J, Rump L C, Walson P D. Liquid biopsies: donor-derived cell-free DNA for the detection of kidney allograft injury. Nat Rev Nephrol. 2021 September; 17(9):591-603. doi: 10.1038/s41581-021-00428-0. Epub 2021 May 24. PMID: 34031575.
Oellerich M, Shipkova M, Asendorf T, Walson P D, Schauerte V, Mettenmeyer N, Kabakchiev M, Hasche G, Gröne H J, Friede T, Wieland E, Schwenger V, Schutz E, Beck J. Absolute quantification of donor-derived cell-free DNA as a marker of rejection and graft injury in kidney transplantation: Results from a prospective observational study. Am J Transplant. 2019 November; 19(11):3087-3099. doi: 10.1111/ajt.15416. Epub 2019 May 28. PMID: 31062511; PMCID: PMC6899936.
Grskovic M, Hiller D J, Eubank L A, Sninsky J J, Christopherson C, Collins J P, Thompson K, Song M, Wang Y S, Ross D, Nelles M J, Yee J P, Wilber J C, Crespo-Leiro M G, Scott S L, Woodward R N. Validation of a Clinical-Grade Assay to Measure Donor-Derived Cell-Free DNA in Solid Organ Transplant Recipients. J Mol Diagn. 2016 November; 18(6):890-902. doi: 10.1016/j.jmoldx.2016.07.003. Epub 2016 Oct. 7. PMID: 27727019.
Huang E, Sethi S, Peng A, et al. Early clinical experience using donor-derived cell-free DNA to detect rejection in kidney transplant recipients. Am J Transplant. 2019; 19: 1663-1670.
Marsh C L, Kurian S M, Rice J C, Whisenant T C, David J, Rose S, Schieve C, Lee D, Case J, Barrick B, Peddi V R, Mannon R B, Knight R, Maluf D, Mandelbrot D, Patel A, Friedewald J J, Abecassis M M, First M R. Application of TruGraf vi: A Novel Molecular Biomarker for Managing Kidney Transplant Recipients With Stable Renal Function. Transplant Proc. 2019 April; 51(3):722-728. doi: 10.1016/j.transproceed.2019.01.054. Epub 2019 Jan. 26. PMID: 30979456.
North P E, Ziegler E, Mahnke D K, Stamm K D, Thomm A, Daft P, Goetsch M, Liang H L, Baker M A, Vepraskas A, Rosenau C, Dasgupta M, Simpson P, Mitchell M E, Tomita-Mitchell A. Cell-free DNA donor fraction analysis in pediatric and adult heart transplant patients by multiplexed allele-specific quantitative PCR: Validation of a rapid and highly sensitive clinical test for stratification of rejection probability. PLoS One. 2020 Jan. 13; 15(1):e0227385. doi: 10.1371/journal.pone.0227385. PMID: 31929557; PMCID: PMC6957190.
Sigdel T K, Archila F A, Constantin T, Prins S A, Liberto J, Damm I, Towfighi P, Navarro S, Kirkizlar E, Demko Z P, Ryan A, Sigurjonsson S, Sarwal R D, Hseish S C, Chan-On C, Zimmermann B, Billings P R, Moshkevich S, Sarwal M M. Optimizing Detection of Kidney Transplant Injury by Assessment of Donor-Derived Cell-Free DNA via Massively Multiplex PCR. J Clin Med. 2018 Dec. 23; 8(1):19. doi: 10.3390/jcm8010019. PMID: 30583588; PMCID: PMC6352163.
Snyder T M, Khush K K, Valantine H A, Quake S R. Universal noninvasive detection of solid organ transplant rejection. Proc Natl Acad Sci USA. 2011 Apr. 12; 108(15):6229-34. doi: 10.1073/pnas.1013924108. Epub 2011 Mar. 28. PMID: 21444804; PMCID: PMC3076856.
Iwijn De Vlaminck et al., Circulating Cell-Free DNA Enables Noninvasive Diagnosis of Heart Transplant Rejection.Sci. Transl. Med.6,241ra77241ra77(2014).DOI:10.1126/scitranslmed.3007803.
Hardy G H. Mendelian Proportions In A Mixed Population. Science. 1908 Jul. 10; 28(706):49-50. doi: 10.1126/science.28.706.49. PMID: 17779291.
Levitsky J, Kandpal M, Guo K, Kleiboeker S, Sinha R, Abecassis M. Donor-derived cell-free DNA levels predict graft injury in liver transplant recipients. Am J Transplant. 2022 February; 22(2):532-540. doi: 10.1111/ajt.16835. Epub 2021 Sep. 24. PMID: 34510731.
Khush K K, Patel J, Pinney S, et al. Noninvasive detection of graft injury after heart transplant using donor-derived cell-free DNA: a prospective multicenter study. Am J Transplant 2019; 19:2889-99.
Hardy G H. MENDELIAN PROPORTIONS I N A MIXED POPULATION. Science. 1908 Jul. 10; 28(706):49-50. Doi: 10.1126/science.28.706.49. PMID: 17779291.
Schutz et al. Detection and quantification of donor cell-free DNA in the circulation of a transplant recipient, U.S. Pat. No. 11,155,872 B2.
Schutz et al. Methods of quantifying cell-free DNA, U.S. Pat. No. 10,570,443.
Rajeevan H, et al. ALFRED: the ALelle FREquency Database. Update. Nucleic Acids Res. 2003 Jan. 1; 31(1):270-1. doi: 10.1093/nar/gkg043. PMID: 12519999; PMCID: PMC165490.

What is claimed is:

1. A method for monitoring, detecting and/or diagnosing a transplant rejection in a patient, comprising the steps of: (a) isolating a cell-free DNA (cfDNA) from a plasma sample from the patient;
(b) enriching a group of target regions in the cfDNA;
wherein the group comprises:
from about 24 to about 384 target regions, and/or
from about 24 to 100 target regions, and/or
from about 24 to about 96 target regions, and/or
from about 24 to about 72 target regions, and/or
from about 24 to about 48 target regions, and/or
wherein each target region comprise a single nucleotide polymorphism (SNP), and/or
wherein the group is predetermined for the patient without
an a priori knowledge of a genotype of the patient, and/or
an a priori knowledge of a genotype of a transplant donor, and/or
wherein the plasma sample is isolated from a whole blood sample from the patient,
and/or
wherein the enriching step (b) comprise:
a ligation step; and/or
a hybridization capture step; and/or
a PCR amplification step; and/or
a molecular inversion probes step; and/or
a selective circularization step; and/or
wherein the enriching step (b) is optionally split into sub-reactions, and
wherein this optional step improves uniformity of the method; and/or
wherein amplification bias is minimized; and/or
wherein all target regions amplify with similar efficiencies;
(c) detecting a sequence encoding the SNP using a master mix;
wherein the master mix is designed to detect N_{SNP};
wherein N_{SNP} is the number of sequences encoding a unique SNP; and/or
wherein N_{SNP} is equal to or greater than the number of color channels on the detection instrument; and/or
wherein a plurality of master mixes are processed simultaneously in a plurality of reactions and each master mix measures a plurality of different SNP; and/or
wherein from about 6 to about 12 master mixes are processed simultaneously in eight reactions; and/or
wherein each master mix measure from about 4 to about 10 different SNPs;
and/or
wherein the sequence is amplified, preferably by a PCR, more preferably by a digital PCR, more preferably by a multiplex digital PCR;
(d) determining a relative copy number for each target region;
(e) identifying donor informative SNPs based on the relative copy number of each target relative to all other targets;
(e) analyzing the donor informative SNP counts across all reactions to determine the total level of donor-derived cfDNA present; and
(h) determining a rejection probability based on thresholds set based on background recipient-derived cfDNA.

2. The method of claim 1,
wherein prior to the sequences being amplified, the method further comprises a cfDNA quality control step,
wherein the quality control step comprises quantification and sizing of the cfDNA; and/or wherein each target region comprise a single nucleotide polymorphism (SNP),
wherein the target regions comprise at least one informative targets; and
wherein each informative target comprise an informative SNP; and
wherein an informative SNP is present in the transplant donor and absent in the transplant patient, and/or
wherein the group of target regions comprise:
at least two informative targets; and/or
at least four informative targets; and/or
at least six informative targets; and/or
at least eight informative targets; and/or
at least ten informative targets; and/or
from about two to about 384 informative targets; and/or
from about four to about 384 informative targets; and/or
from about six to about 384 informative targets; and/or
from about eight to about 384 informative targets; and/or
from about ten to about 384 informative targets; and/or
wherein the number of informative targets is achieved in an assay comprising:
at most 24 target regions; and/or
at most 48 target regions; and/or
at most 72 target regions; and/or
at most 96 target regions; and/or
wherein the SNP has a minor allele frequency in the human population of:
from about 0.1 to 0.39, from about 0.11 to 0.39, from about 0.12 to 0.39, from about 0.13 to 0.39, from about 0.14 to 0.39, from about 0.15 to 0.39, from about 0.16 to 0.39, from about 0.17 to 0.39, from about 0.18 to 0.39, from about 0.19 to 0.39, from about 0.2 to 0.39, from about 0.21 to 0.39, from about 0.22 to 0.39, from about 0.23 to 0.39, from about 0.24 to 0.39, from about 0.25 to 0.39, from about 0.26 to 0.39, from about 0.27 to 0.39, from about 0.28 to 0.39, from about 0.29 to 0.39, from about 0.3 to 0.39, from about 0.31 to 0.39, from about 0.32 to 0.39, from about 0.33 to 0.39, from about 0.34 to 0.39, from about 0.35 to 0.39, from about 0.36 to 0.39, from 0.37 to 0.39, and/or from 0.38 to 0.39; and/or
from about 0.1 to 0.39, from about 0.1 to 0.38, from about 0.1 to 0.37, from about 0.1 to about 0.36, from about 0.1 to about 0.35, from about 0.1 to about 0.34, from about 0.1 to about 0.33, from about 0.1 to about 0.32, from about 0.1 to about 0.31, from about 0.1 to about 0.30, from about 0.1 to about 0.29, from about 0.1 to about 0.28, from about 0.1 to about 0.27, from about 0.1 to about 0.26, from about 0.1 to about 0.25, from about 0.1 to about 0.24, from about 0.1 to about 0.23, from about 0.1 to about 0.22, from about 0.1 to about 0.21, from about 0.1 to about 0.20, from about 0.1 to about 0.19, from about 0.1 to about 0.18, from about 0.1 to about 0.17, from about 0.1 to about 0.16, from about 0.1 to about 0.15, from about 0.1 to about 0.14, from about 0.1 to about 0.13, from about 0.1 to about 0.12, and/or from about 0.1 to about 0.11; and/or
from about 0.1 to 0.39, from about 0.11 to 0.38, from about 0.12 to 0.37, from about 0.13 to about 0.36, from about 0.14 to about 0.35, from about 0.15 to about 0.34, from about 0.16 to about 0.33, from about 0.17 to about 0.32, from about 0.18 to about 0.31, from about 0.19 to about 0.30, from about 0.2 to about 0.29, from about 0.21 to about 0.28, from about 0.22 to about 0.27, from about 0.23 to about 0.26, and/or from about 0.24 to about 0.25; and/or
about 0.3, about 0.29, about 0.293, or about 0.2929; and/or
about 1−\sqrt{0.5};
wherein the SNP value is selected from a publication from a regional or a national institution or organization; and/or
wherein the SNP value is selected from a publication from the National Center for Biotechnology Information, National Human Genome Research Institute, 1000 Genome Project, The Genome Aggregation Database (gnomAD), HapMap project, SNPedia, UK Biobank, and/or the likes; and/or
wherein the organ and/or tissue transplanted is a whole or a part of:
a kidney, liver, heart, lung, pancreas, intestine, stomach, cornea, heart valve, skin, bone marrow, bone, muscles, blood vessels, nerves, connective tissues, tendons, and/or combinations thereof; or
a kidney, liver, heart, lung, pancreas, intestine, and/or combinations thereof; or
a kidney, liver, heart, and/or combinations thereof; or
a kidney, or
a liver, or
a heart; and/or
wherein the transplant donor:
is not a merozygote twin of the patient; and/or
does not have cancer at the time of transplant; and/or
is not pregnant at the time of transplant; and/or
wherein the transplant donor is:
a full biological sibling of the patient; or
a biological parent or biological child of the patient; or
a half biological sibling of the patient; and/or
biologically unrelated to the patient; and/or
further comprising isolating buffy coat gDNA in parallel for host genotyping,
wherein the buffy coat gDNA sample is processed through the same set of multiplex reactions to determine the host genotype, and/or
wherein donor informative SNPs are identified based on a buffy coat gDNA genotype profile, and/or
wherein a buffy coat dPCR signal is used in a background subtraction step to reduce noise.

3. A composition for monitoring a transplant rejection in a patient, wherein the composition comprise a plurality of primers,
wherein the primer encodes a sequence complementary a target region, and
wherein the plurality of primers has a total binding capacity to a group of target regions;
wherein the group of target regions has a total of:
from about 24 to about 384 target regions, and/or
from about 24 to 100 target regions, and/or
from about 24 to about 96 target regions, and/or
from about 24 to about 72 target regions, and/or
from about 24 to about 48 target regions, and/or
wherein each target region comprise a single nucleotide polymorphism (SNP), and/or;
wherein each SNP has a minor allele frequency in the world human population of:
from about 0.1 to 0.39, from about 0.11 to 0.39, from about 0.12 to 0.39, from about 0.13 to 0.39, from about 0.14 to 0.39, from about 0.15 to 0.39, from about 0.16 to 0.39, from about 0.17 to 0.39, from about 0.18 to 0.39, from about 0.19 to 0.39, from about 0.2 to 0.39, from about 0.21 to 0.39, from about 0.22 to 0.39, from about 0.23 to 0.39, from about 0.24 to 0.39, from about 0.25 to 0.39, from about 0.26 to 0.39, from about 0.27 to 0.39, from about 0.28 to 0.39, from about 0.29 to 0.39, from about 0.3 to 0.39, from about 0.31 to 0.39, from about 0.32 to 0.39, from about 0.33 to 0.39, from about 0.34 to 0.39, from about 0.35 to 0.39, from about 0.36 to 0.39, from 0.37 to 0.39, and/or from 0.38 to 0.39; and/or
from about 0.1 to 0.39, from about 0.1 to 0.38, from about 0.1 to 0.37, from about 0.1 to about 0.36, from about 0.1 to about 0.35, from about 0.1 to about 0.34, from about 0.1 to about 0.33, from about 0.1 to about 0.32, from about 0.1 to about 0.31, from about 0.1 to about 0.30, from about 0.1 to about 0.29, from about 0.1 to about 0.28, from about 0.1 to about 0.27, from about 0.1 to about 0.26, from about 0.1 to about 0.25, from about 0.1 to about 0.24, from about 0.1 to about 0.23, from about 0.1 to about 0.22, from about 0.1 to about 0.21, from about 0.1 to about 0.20, from about 0.1 to about 0.19, from about 0.1 to about 0.18, from about 0.1 to about 0.17, from about 0.1 to about 0.16, from about 0.1 to about 0.15, from about 0.1 to about 0.14, from about 0.1 to about 0.13, from about 0.1 to about 0.12, and/or from about 0.1 to about 0.11; and/or
from about 0.1 to 0.39, from about 0.11 to 0.38, from about 0.12 to 0.37, from about 0.13 to about 0.36, from about 0.14 to about 0.35, from about 0.15 to about 0.34, from about 0.16 to about 0.33, from about 0.17 to about 0.32, from about 0.18 to about 0.31, from about 0.19 to about 0.30, from about 0.2 to about 0.29, from about 0.21 to about 0.28, from about 0.22 to about 0.27, from about 0.23 to about 0.26, and/or from about 0.24 to about 0.25; and/or
about 0.3, about 0.29, about 0.293, or about 0.2929; and/or
about 1−\sqrt{0.5}.

4. The composition of claim 3,
wherein the group of target regions comprise at least one informative targets; and/or
wherein each informative target comprise an informative SNP; and/or
wherein an informative SNP is present in a transplant donor and absent in the transplant patient;
wherein the group of target regions comprise:
at least two informative targets; and/or
at least four informative targets; and/or
at least six informative targets; and/or
at least eight informative targets; and/or
at least ten informative targets; and/or
from about two to about 384 informative targets; and/or
from about four to about 384 informative targets; and/or
from about six to about 384 informative targets; and/or
from about eight to about 384 informative targets; and/or
from about ten to about 384 informative targets; and/or
wherein the number of informative targets is achieved in an assay comprising at most:
about 24 target regions; and/or
about 48 target regions; and/or
about 72 target regions; and/or
about 96 target regions; and/or
wherein the informative SNP is bi-allelic and/or
wherein the sum of the two predominant allele frequency of the informative SNP is about 99% or greater in a population;
wherein the plurality of primers is predetermined for the patient without an a priori knowledge of a genotype of the patient, and/or
wherein the plurality of primers is predetermined for the patient without an a priori knowledge of a genotype of the transplant donor, and/or wherein the organ and/or tissue transplanted is a whole or a part of:
a kidney, liver, heart, lung, pancreas, intestine, stomach, cornea, heart valve, skin, bone marrow, bone, muscles, blood vessels, nerves, connective tissues, tendons, and/or combinations thereof; or
a kidney, liver, heart, lung, pancreas, intestine, and/or combinations thereof; or
a kidney, liver, heart, and/or combinations thereof; or
a kidney, or
a liver, or
a heart; and/or wherein the transplant donor:
is not a merozygote twin of the patient; and/or
does not have cancer at the time of transplant; and/or
is not pregnant at the time of transplant; and/or
wherein the transplant donor is:
a full biological sibling of the patient; or
a biological parent or biological child of the patient; or
a half biological sibling of the patient; or
biologically unrelated to the patient; and/or
wherein the transplant organ or tissue is from a human; and/or
wherein the composition further comprises one or more of:
(a) a DNA polymerase; and/or
(b) a RNase H; and/or
(c) a dNTP mixture; and/or
(d) a buffer; and/or
(e) a detection probe
(f) a magnesium compound; wherein (a)-(f) are stored in separate compartments prior to use;
wherein each detection probe comprises:
a fluorophore and optionally a quencher,
wherein the fluorophore and optionally the quencher is conjugated to the primer;
wherein each detection probe encodes a sequence complementary to the primer;
wherein the number of unique detection probes specific for each target region is between 1 and nₚ, wherein nₚ is selected from the list consisting of 1, 2, 3, 4, 5, 6, 7, 8, 9, 10, 11, and 12; and/or
wherein the number of unique detection probes specific for the group of target regions in a sample volume is between 1 and Np, wherein Np is selected from the list of 1, 4, 8, 12, 16, 20, 24, 28, 32, 36, 40, 44, 48, 52, 56, 60, 64, 68, 72, 76, 80, 84, 88, 92 and 96 or 2, 3, 4, 5, 6, 7, 8, 9, 10, 11, 12, 13, 14, 15, 16, 17, 18, 19, and 20;
wherein a detection probe is conjugated to a first fluorophore and optionally conjugated to a first quencher;
wherein combination of the emission color and emission intensity for each detection probe is unique;
wherein the fluorophore is:
optionally selected from a group of ABY, Alexa Fluor 350, Alexa Fluor 405, Alexa Fluor 488, Alexa Fluor 532, Alexa Fluor 546, Alexa Fluor 555, Alexa Fluor 568, Alexa Fluor 594, Alexa Fluor 647, AlexaFluor 680, Alexa Fluor 750, ATTO 425, ATTO 550, ATTO 590, Cyan500, Cy3, Cy5, Cy5.5, Texas Red, Fluorescein (FITC), 6-FAM, 5-FAM, HEX, JOE, TAMRA, ROX, BODIPY FL, Pacific Blue, Pacific Green, Coumarin, Oregon Green, Pacific Orange, VIC, LC610, CFR610, JA270, LC640, JUN, Trimethylrhodamine (TRITC), Cal Fluor dyes, Quasar dyes, DAPI, APC, Cyan Fluorescent Protein (CFP), Green Fluorescent Protein (GFP), Red Fluorescent Protein (RFP), Phycoerythin (PE), quantum dots (for example, Qdot 525, Qdot 565, Qdot 605, Qdot 705, Qdot 800), Cal Fluor Orange 560, Cal Fluor Red 590, Cal Fluor Red 610, Cal Fluor Red 635, Quasar 570, Quasar 670, Quasar 705, derivatives thereof and combinations thereof; and/or
preferably selected from a group consisting of ATTO 425, FAM, HEX, TAMRA, Texas Red, Cy5, ATTO 590, ROX, or Cy5.5, derivatives thereof, and combinations thereof; and/or
more preferably selected from a group of ATTO 425, FAM, HEX, Texas Red, Cy5, Cy5.5, derivatives thereof, and combinations thereof; and/or
wherein the quencher is selected from the group consisting of:
TAMRA, BHQ-1, BHQ-2, BHQ-3, IowaBlack FQ, ZEN, or Dabcy,
derivatives thereof, and combinations thereof; and/or wherein the composition, once combined with a cell free DNA (cfDNA) from a plasma sample from the patient, can monitoring, detecting and/or diagnosing a transplant rejection in a patient; and/or
wherein the composition can amplify target regions, preferably by a PCR, more preferably by a digital PCR, more preferably by a multiplex digital PCR.

5. A kit for detecting, analyzing, evaluating, screening for, prognosing, diagnosing, and/or monitoring, a condition in the patient comprising:
the composition of claim 3,
wherein the primers and one or more of (a)-(f) are stored separately in a container; and/or
wherein the primers and two or more of (a)-(f) are pre-mixed and stored together in a container; and
an instruction.

6. A kit for detecting, analyzing, evaluating, screening for, prognosing, diagnosing, and/or monitoring, a condition in the patient comprising:
the composition of claim 4,
wherein the primers and one or more of (a)-(f) are stored separately in a container; and/or
wherein the primers and two or more of (a)-(f) are pre-mixed and stored together in a container; and
an instruction.

7. The kit of claim 5, wherein the instruction is for the method of claim 1.

8. The kit of claim 5, wherein the instruction is for the method of claim 2.

9. The kit of claim 6, wherein the instruction is for the method of claim 1.

10. The kit of claim 6, wherein the instruction is for the method of claim 2.
